(19) United States
(12) Patent Application Publication
(10) Pub. No.: US 2026/0071187 A1
(43) Pub. Date: Mar. 12, 2026

(54) MOLECULARLY CLEAVABLE BIOINK FORMULATION

(71) Applicant: THE BRIGHAM AND WOMEN'S HOSPITAL, INC., Boston, MA (US)

(72) Inventor: Yu Shrike Zhang, Boston, MA (US)

(21) Appl. No.: 18/869,925

(22) PCT Filed: Jun. 2, 2023
(86) PCT No.: PCT/US2023/067812

§ 371 Date: Nov. 27, 2024

Related U.S. Application Data
(60) Provisional application No. 63/348,062, filed on Jun. 2, 2022.

(51) Int. Cl. C12N 5/071; B29C 64/129; B29C 64/30; B29C 71/00; B29K 105/00; B29L 31/00; B33Y 10/00; B33Y 40/20; B33Y 70/10; B33Y 80/00; C08F 291/12; C09D 4/06; C09D 151/08; C12N 5/077; C12N 5/0797

(52) U.S. Cl. CPC C12N 5/0671; B29C 64/129; B29C 64/30; B29C 71/00; B33Y 10/00; B33Y 40/20; B33Y 70/10; B33Y 80/00; C08F 291/12; C09D 4/06; C09D 151/08; C12N 5/0623; C12N 5/0658; B29K 2005/00; B29K 2089/00; B29K 2105/0002; B29L 2031/753; C12N 2501/73; C12N 2513/00; C12N 2533/54; C12N 2533/70

(57) ABSTRACT

A bioink formulation for digital light processing bioprinting comprising a mixture of a biocompatible cleavable polymer precursor, a biocompatible non-cleavable polymer precursor, and a photoinitiator is described. Three-dimensional (3D) objects prepared using these bioink formulations are also described. In addition, a method of 3D bioprinting is described. The method includes providing a bioink formulation in a 3D bioprinter vat; repeatedly photoactivating the biocompatible photoactive polymer precursors in the 3D bioprinter vat on a build plate immersed in the vat to form a 3D bioprinted object comprising polymers having a series of predefined shapes across the vertical direction based on a set of sliced images; and treating the 3D bioprinted object with an agent that cleaves chemical bonds within the cleavable polymer.

### CONTINUING APPLICATION DATA

This application claims the benefit of U.S. Provisional Application Ser. No. 63/348,062, filed Jun. 2, 2022, the disclosure of which is incorporated by reference herein.

### STATEMENT REGARDING FEDERALLY SPONSORED RESEARCH

This invention was made with government support under Grant Numbers R21EB026175, R21EB025270, R00CA201603, and R01EB028143, awarded by the National Institutes of Health, and Grant Number CBET-EBMS-123859, awarded by the National Science Foundation. The Government has certain rights in this invention.

### BACKGROUND

Additive biomanufacturing, commonly known as three-dimensional (3D) bioprinting, has drawn on continuously increasing attention in tissue biofabrication for a range of applications. Mota, C., et al. Chem. Rev. 120, 10547-10607 (2020). In particular, proper integration of 3D bioprinting with meticulously designed bioinks is necessary to meet multifactorial mechanical and physiological requirements optimized for tissue generation. To date, significant progress has been made towards producing structurally sophisticated constructs featuring tissue-mimicking shapes and geometries using various 3D bioprinting modalities. Jorgensen, A. M. et al., Chem. Rev. 120, 11093-1t 127 (2020). Notably, 3D bioprinting using the digital light processing (DLP)-based approach oftentimes exhibits a superior performance in terms of both printing speed as well as structural complexity compared to other bioprinting methods, such as extrusion-based techniques. Lim, K. S. et al., Chem. Rev. 120, 10662-10694 (2020). For example, bioprinting of hydrogel models of a distal lung-mimicking construct and vessel-like microchannel-embedded constructs, among others, were recently reported through the use of DLP-based (bio) printing. Grigoryan, B. et al., Science 364, 458-464 (2019).

Although structural complexity plays a vital role in tissue recapitulation, physiological microenvironments are also essential to be considered to attain proper tissue-specific functions. West-Livingston, L. N. et al., Chem. Rev. 120, 11056-11092 (2020). In fact, very little optimization work has been carried out to formulate enabling bioinks for DLP bioprinting, which should meet the mechanical demand of printing fidelity while simultaneously satisfying the biological requirements for the loaded cell types. Li, W. et al., Adv. Healthc. Mater. 9, 2000156 (2020). In particular, it remains highly challenging to construct soft organ-mimics such as the brain and the liver. Matai, I. et al., Biomaterials 226, 119536 (2020). As a matter of fact, the prerequisites in bioink development for soft-tissue biofabrication are normally mutually exclusive across the different bioprinting modalities. On the one hand, bioinks with strong mechanical properties can aid in filament deposition in extrusion bioprinting or layer-by-layer lifting in DLP bioprinting. Schwab, A. et al., Chem. Rev. 120, 11028-11055 (2020). Nevertheless, the stiff bioink networks result in limited cellular functions, including but not limited to cell spreading, proliferation, and differentiation, for cells that are of soft tissues in origin. Liu, W. et al. Adv. Healthc. Mater. 6, 1601451 (2017). On the other hand, the mechanical properties of bioinks that are compatible with soft tissue-derived cells are usually insufficient to facilitate the bioprinting process, especially when volumetric constructs are desired. Ouyang, L. et al., Sci. Adv. 6, eabc5529 (2020). This dilemma is well-exemplified by the vast body of currently existing reports on DLP bioprinting; high-fidelity, volumetrically sophisticated structures with limited bioactivities are obtainable when the (bio)ink mechanics are high, Grigoryan, B. et al., Science 364, 458-464 (2019), whereas soft bioinks with good cellular activities would only allow creation of planar or pseudo-3D tissue constructs. Tang, M. et al. Cell Res. 30, 833-853 (2020). Therefore, the lack of cytocompatible yet mechanically tunable bioink design remains as a major inhibitor for further applications of DLP bioprinting of truly 3D, structurally and biologically relevant tissue constructs.

To overcome this obstacle, some strategies have been proposed through modulating different photocrosslinking ratios in DLP-based bioprinting, where soft constructs with mechanical properties as low as 10 kPa were obtained. Further exploration has been made via the utilization of a fluid support, where a support fluid immiscible with bioinks were used to provide extra buoyant forces for printing. Beh, C. W. et al., Biomaterials 276, 121034 (2021). Nevertheless, the lowest mechanical properties of bioprinted constructs with this method were still limited to ˜7 kPa, in addition to the concerns associated with the extremely complicated instrumentation setup. Furthermore, uncertainty remains as to the instable bioprinting process due to the presence of the liquid interface, as well as the unwanted contamination by the immiscible support fluid. These strategies have achieved a few of the above requirements, such as volumetric printability and some degrees of cytocompatibility. Other progress has been made with a bioink of complementary thermoreversible gelatin network in extrusion-based bioprinting, whereby the bioprinted constructs could meet biological requirements of loaded cells via the sacrifice of the gelatin molecules. Ouyang, L. et al., Sci. Adv. 6, eabc5529 (2020). Despite many efforts been exerted so far, the reconstitution of ultrasoft tissues through DLP bioprinting still needs notable progress.

### SUMMARY

Digital light processing bioprinting favors biofabrication of tissues with improved structural complexity. However, soft-tissue fabrication with this method remains a challenge to balance the physical performances of the bioinks for high-fidelity bioprinting and suitable microenvironments for the encapsulated cells to thrive. The inventors have developed a molecular cleavage approach, where hyaluronic acid methacrylate (HAMA) is mixed with gelatin methacryloyl to achieve high-performance bioprinting, followed by selectively enzymatic digestion of HAMA, resulting in tissue-matching mechanical properties without losing the structural complexity and fidelity. The method allows cellular morphological and functional improvements across multiple bioprinted tissue types featuring a wide range of mechanical stiffness, from the muscles to the brain, the softest organ of the human body. This platform endows us to biofabricate mechanically precisely tunable constructs to meet the biological function requirements of target tissues, potentially paving the way for broad applications in tissue and tissue model engineering, among others.

In one aspect, a bioink formulation for three-dimensional bioprinting is provided. The formulation includes a mixture of a biocompatible cleavable polymer precursor, a biocompatible non-cleavable polymer precursor, and a photoinitiator.

In some embodiments, the non-cleavable polymer precursor is gelatin methacryloyl (GelMA). In further embodiments, the GelMA has a concentration ranging from 2.5% to 7.5% by weight. In additional embodiments, the cleavable polymer precursor is a hyaluronic acid derivative. In further embodiments, the molecular weight (Mw) of the hyaluronic acid derivative ranges from about 10 kDa to about 1,500 kDa. In yet further embodiments, the Mw of the hyaluronic acid derivative ranges from about 75 kDa to about 125 kDa. In additional embodiments, the hyaluronic acid derivative is hyaluronic acid methacrylate (HAMA), and in yet further embodiments the HAMA has a concentration ranging from 0.5% to 5% by weight.

In some embodiments, the non-cleavable polymer precursor is GelMA and the cleavable polymer precursor is HAMA. In further embodiments, the GelMA has a concentration ranging 2% to 6% by weight, and the HAMA has a concentration ranging from 1% to 2.5% by weight. In additional embodiments, the bioink formulation further comprises cells.

An additional aspect of the invention provides a method of three-dimensional (3D) bioprinting. The method can include the steps of providing a biocompatible cleavable polymer precursor, a biocompatible non-cleavable polymer precursor, and a photoinitiator in a 3D bioprinter vat; repeatedly photoactivating the biocompatible polymer precursors in the 3D bioprinter vat on a build plate immersed in the vat to form a 3D bioprinted object comprising polymers having a series of predefined shapes across the vertical direction based on a set of sliced images; and treating the 3D bioprinted object with an agent that cleaves chemical bonds within the cleavable polymer.

In some embodiments, the agent is an enzyme. In further embodiments, the enzyme is a glycosidase or a protease.

In some embodiments, the non-cleavable polymer precursor is gelatin methacryloyl (GelMA), while in additional embodiments the cleavable polymer precursor is hyaluronic acid methacrylate (HAMA). In further embodiments the mixture of biocompatible polymers further comprises cells.

In some embodiments, the 3D bioprinted object is a tissue construct. In one embodiment, the tissue construct is a liver tissue construct and the mix of polymers further comprises hepatocytes. In another embodiment, the tissue construct is a muscle tissue construct and the mix of polymers further comprises myoblasts. In a further embodiment, the tissue construct is a brain tissue construct and the mix of polymers further comprises neural progenitor cells.

Another aspect of the invention provides a three-dimensional (3D) object prepared from a bioink formulation is provided. The 3D object can be prepared by providing a bioink formulation comprising a mixture of a biocompatible cleavable polymer precursor, a biocompatible non-cleavable polymer precursor, and a photoinitiator in a 3D bioprinter vat; repeatedly photoactivating the bioink formulation in the 3D bioprinter vat on a build plate immersed in the vat to form a 3D bioprinted object comprising a mix of polymers having a series of predefined shapes across the vertical direction based on a set of sliced images; and treating the 3D bioprinted object with an agent that cleaves chemical bonds within the cleavable polymer of the mix of polymers.

In some embodiments, the 3D bioprinted object is a soft tissue construct. In further embodiments, the 3D bioprinted object further comprises cells. In additional embodiments, the non-cleavable polymer is gelatin methacryloyl (GelMA), while in yet further embodiments the cleavable polymer is hyaluronic acid methacrylate (HAMA).

### BRIEF DESCRIPTION OF THE FIGURES

FIG. 1 provides a flow chart showing the steps involved in a method of digital light processing 3D bioprinting.

FIG. 2 provides a schematic view of the in-house-built bottom-up configuration DLP-based 3D bioprinter.

FIGS. 3a-3d provide (3a) a schematic representation of the DLP-based 3D bioprinting with conventional, pure GelMA bioinks, where bioprinting would fail because of the insufficient mechanical properties when the bioinks are used at low concentrations; (3b) a schematic representation of the DLP-based 3D bioprinting showing high-fidelity fabrication using the GelMA bioinks homogeneously mixed with HAMA; (3c) describes the enzymatic digestion procedure of the GelMA/HAMA-bioprinted constructs with elevated Hase concentration and treatment duration, enabling increased cleavage of HA, reducing matrix stiffness, and promoting cell spreading and functions; (3d) describes that tissue-mimics bioprinted with the GelMA/HAMA bioinks could be obtained by precisely controlling the post-bioprinting digestion of the HAMA component to achieve target tissue-matching mechanical properties across a wide range.

FIGS. 4a-4f provide (4a) printability maps and representative images of 3D-printed cubes using diverse enzyme-digestible inks, covering 2.5%, 5.0%, or 7.5% by weight GelMA, and 0 to 3% by weight HAMA (M_{w}=10, 60, 100, 200, 500, or 1500 kDa); (4b) represents compressive moduli of samples fabricated from GelMA/HAMA inks across various formulations. n=3; one-way ANOVA; *p<0.05, **p<0.01, ***p<0.001 (compared with the respective control groups of 0% HAMA). Data are presented as mean values±SDs; (4c-f) provide photographs showing 3D-printed constructs featuring sophisticated shapes and internal structures, using the GelMA/HAMA ink of 5.0%/3.0% by weight (M_{w} of HAMA=100 kDa) and corresponding digital models used for printing; (4c) represents pyramid in the side view; (4d) represents gyroid in the side view; (4e) represents cuboid containing a spiral, perfusable channel in the top view and side view. f Torus knot (the digital design was obtained from 11) in the top view and side view; (4a) and (4c-4f) provide images that are representatives of n=3 independent experiments. G GelMA, H HAMA.

FIGS. 5a-5e provide (Sa) 3D surface plots showing the effects of different parameters on mechanical properties of the final constructs made from 3.0% HAMA by weight in combination with different concentrations of GelMA and Hase; (5b) represents comparison plot between predicted and experimental moduli of Hase-digested constructs; (5c) parameter maps showing the relationships between compressive moduli and bioink formulations, including GelMA concentration, Hase concentration, and the digestion time; (5d) represents compressive moduli of constructs printed with different ink formulations to achieve tissue-matching mechanics post-enzymatic digestion, where the digestion parameters were determined from the mathematical model. n=3; one-way ANOVA; **p<0.01 (compared with the respective groups of before digestion). Data are presented as mean values±SDs; (5c) provides 3D tissue-mimics printed with 2.5%, 5.0%, 5.0%, or 7.5% GelMA by weight containing 1.5% or 3.0% HAMA by weight to emulate the brain, liver, lung, or heart, respectively. Bottom rows show photographs of the printed structurally sophisticated constructs before and after different parameters of Hase digestion and the corresponding SEM images. Images are representatives of n=3 independent experiments.

FIGS. 6a-6i provide (6a) micrographs showing MHC (green) staining of C2C12 cells cultured in the bioprinted constructs from GelMA/HAMA (7.5%/1.5% by weight) without or with Hase digestion (1000 U mL⁻¹, 24 h) at the days 7 and 14 after myogenic differentiation; (6b) describes corresponding quantitative results of the MHC+ cells and fusion index; (6c) provides micrographs showing live (green)/dead (red) staining of HepG2/C3A cells encapsulated in constructs bioprinted from 10% GelMA, and GelMA/HAMA (5.0%/1.5% by weight) without or with Hase digestion (1000 U mL⁻¹, 24 h) at the days 1, 3, and 7 of culture; (6d) provides corresponding quantitative analyses of the percentages of live/dead cells; (6e) Quantitative results of MTS assay showing metabolic activities of the HepG2/C3A cells; (6f, 6g) represent quantitative results of ALB- and urea-secretion levels of the HepG2/C3A cells grown in the bioprinted GelMA/HAMA (5.0%/1.5%) constructs without or with Hase digestion (1000 U mL⁻¹, 24 h) after 5, 10, and 15 days; (6h) gene expression profiles showing expression levels of MKI67, ALB, AFP, and CASP8 for the HepG2/C3A cells grown in the bioprinted GelMA/HAMA (5.0%/1.5%) constructs without or with Hase digestion (1000 U mL⁻¹, 24 h) after 7 and 14 days. All gene expression fold changes are relative to the corresponding expressions of the GH group; (6i) represents confocal immunofluorescence images showing staining for ALB (green) and E-cadherin (red), or CYP1A2 (red), or CYP3A4 (green), with nuclei counterstaining (blue) of the cells cultured in the bioprinted GelMA/HAMA (5.0%/1.5%) constructs without or with Hase digestion (1000 U mL⁻¹, 24 h) after 14 days. Images are representatives of n=3 independent experiments. FIGS. 6a and b, c and d, and f-h, n=3; e n=9; one-way ANOVA; *p<0.05, **p<0.01, ***p<0.001 (FIGS. 6b and f-h, compared with the group of GH; FIGS. 6d and e, compared with the group of GM); #p<0.05, ##p<0.01, ##p<0.001 (FIG. 6b, compared with the corresponding results of day 7 in the same group; FIGS. 6d and e, compared with the group of GH; FIG. 6g compared with the corresponding results of day 5 in the same group). Data are presented as mean values±SDs. GM indicates the hydrogel made of 10% GelMA, GH is composed of GelMA/HAMA (7.5%/1.5% by weight for skeletal muscle tissue, 5.0%/1.5% by weight for hepatic tissue), and Hase is the group of GelMA/HAMA treated with Hase (1000 U mL⁻¹) for 24 h. MHC myosin skeletal heavy chain, ALB albumin.

FIGS. 7a-7i provide (7a) a timeline of 3D bioprinting of SNaPs and their subsequent culture protocols. The bioink was GelMA/HAMA (2.5%/1.5% by weight), followed by 500 or 1000 U mL⁻¹ of Hase digestion for 24 h post-bioprinting; (7b) represents fluorescence micrographs of SNaPs cultured in GM and GH constructs without or with Hase digestion (500 or 1000 U mL⁻¹, 24 h), stained for NESTIN (red), Ki67 (green), and nuclei counterstaining (blue) at days 1, 2, and 3 after digestion; (7c) represents corresponding quantitative analyses of the percentages of Ki67+ nuclei in the four groups; (7d) Quantitative analyses of the percentages of live/dead cells obtained from live/dead staining images; (7e) describes gene expression clusters for SNaPs after 4 weeks of differentiation (31 days after digestion) in GM and Hase-1000 digested constructs; (7f) represents PCA of gene expression values derived from whole-transcriptome sequencing data for SNaPs after 4 weeks of differentiation in GM and Hase-1000 digested constructs; (7g) provides total gene numbers of upregulation and downregulation for SNaPs after 4 weeks of differentiation in GM and Hase-1000 digested constructs; (5h) represents volcano plot of transcriptional landscape comparing SNaPs after 4 weeks of differentiation in GM and Hase-1000 digested constructs; (7i) represents K-means clusters of genes of the KEGG enrichment analyses for SNaPs after 4 weeks of differentiation in GM and Hase-1000 digested constructs. FIGS. 7b and c, and d n=3; one-way ANOVA; *p<0.05, **p<0.01(compared with the group of GM), #p<0.05, ##p<0.01, ###p<0.001 (compared with the corresponding results of day 1 in the same group). Data are presented as mean values±SDs. GM indicates the hydrogel made of 10% GelMA by weight. GH is composed of GelMA/HAMA (2.5%/1.5% by weight for brain-like tissue), and Hase-500 and Hase-1000 are the groups of GelMA/HAMA treated with Hase (500 and 1000 U mL⁻¹, respectively) for 24 h.

FIG. 8a-8g provide (8a & 8b) confocal fluorescence micrographs showing immunostaining results of (8a) protein markers representing neurons (SNaPs, green), astrocytes (GFAP, magenta), and neural progenitor cells (NESTIN, red) after 4 weeks of differentiation; and (8b) neuronal markers including neurites (TUJ1, green) and synaptic vesicle proteins (synaptophysin, red). In all cases, the bioink was GelMA/HAMA (2.5%/1.5% by weight), followed by 1000 U mL⁻¹ of Hase digestion for 24 h post-bioprinting; (8c) represents optical image showing the 4-week differentiated mini-brain placed on top of the MEA, with maintenance of the overall bioprinted brain shape similar to that shown in FIG. 5e; (8d) represents high-magnification fluorescence micrographs showing SNaPs after 4 weeks of differentiation directly in contact with the MEA surface and representative heatmaps of active electrodes; (8e) describe electrophysiological properties of SNaP-derived neurons in the bioprinted mini-brains with 10% by weight GelMA and GelMA/HAMA (2.5%/1.5% by weight), followed by 1000 U mL⁻¹ of Hase digestion for 24 h post-bioprinting at 1-4 weeks after differentiation: the percentages of active MEA electrode plateaus; the mean firing rates; the numbers of bursts; and the network burst frequencies. n=4; one-way ANOVA; *p<0.05, **p<0.01, ***p<0.001 (compared with the group of GM). Data are presented as mean values±SDs; (8f & 8g) describe electrophysiological property changes of SNaP-derived neurons in the bioprinted mini-brains post-enzymatic digestion, without or with treatment of (8f) AMPA receptor-antagonist NBQX (10 μM) or (8g) NMDA receptor-antagonist D-APV (50 μM) at 4 weeks after culturing in the MEA plates. n=4; two-tailed student's t-test; *p<0.05, **p<0.01, ***p<0.001 (compared with the group of DMSO). Data are presented as mean values±SDs. GM indicates the hydrogel made of 10% GelMA. Hase-1000 indicates the groups of GelMA/HAMA treated with Hase (1000 U mL⁻¹) for 24 h.

### DETAILED DESCRIPTION

A bioink formulation for three-dimensional (3D) bioprinting comprising a mixture of a biocompatible cleavable polymer precursor, a biocompatible non-cleavable polymer precursor, and a photoinitiator is provided. Three-dimensional objects prepared using these bioink formulations are also provided. In addition, a method of 3D bioprinting is provided. The method includes providing a bioink formulation in a 3D bioprinter vat; repeatedly photoactivating the biocompatible polymer precursors in the 3D bioprinter vat on a build plate immersed in the vat to form a 3D bioprinted object comprising polymers having a series of predefined shapes across the vertical direction based on a set of sliced images; and treating the 3D bioprinted object with an agent that cleaves chemical bonds within the cleavable polymer.

### Definitions

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting the application as a whole. For example, as used in this specification and the appended claims, the singular forms “a”, “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form. The conjunctive phrase “and/or” indicates that either or both of the items referred to can be present.

The phrase “consisting essentially of” means that the composition or method may include additional ingredients and/or steps, but only if the additional ingredients and/or steps do not materially alter the basic and novel characteristics of the claimed composition or method.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 11/2, and 43/4 This applies regardless of the breadth of the range.

As used herein, the term “about” means±10% of the recited value.

A weight percent (wt. %) of a component, unless specifically stated to the contrary, is based on the total weight of the formulation or composition in which the component is included.

“Biocompatible” as used herein, refers to the capability of a material to be integrated into a biological system without harming or being rejected by the system. Examples of harm include inflammation, infection, fibrotic tissue formation, cell death, or thrombosis. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

A “subject,” as used herein, can be any animal, and may also be referred to as the patient. Preferably the subject is a vertebrate animal, and more preferably the subject is a mammal, such as a research animal (e.g., a mouse or rat) or a domesticated farm animal (e.g., cow, horse, pig) or pet (e.g., dog, cat). In some embodiments, the subject is a human.

### Molecularly Cleavable Bioink Formulations

In one aspect, the present invention provides a bioink formulation for three-dimensional (3D) bioprinting comprising a mixture of a biocompatible cleavable polymer precursor, a biocompatible photoactive non-cleavable polymer precursor, and a photoinitiator. The bioink provides the material that can be used to print the 3D object, and can also be referred to as a bioresin.

Three-dimensional printing (also known as additive manufacturing) is a method that takes information of the surface shape of a 3D object, models it using computer-aided design/computer-aided manufacturing (CAD/CAM) software, and then slices that model into multiple layers, creating a stacked cross-sectional version of the 3D object. This information is then fed into a 3D printer capable of fabricating the 3D object using an additive or subtractive manufacturing process for constructing three-dimensional single or multi-layered structures disposed on a substrate. Examples of 3D printing methods include stereolithography, direct ink writing, viscous solution printing, and digital light processing.

Digital light processing (DLP) 3D printing is a vat polymerization technique. The DLP process subjects a vat of this liquid resin to high-intensity light from a projector, which selectively cures the resin to a build platform in a layer-by-layer process. The printer's build platform is submerged in a vat of liquid resin, and a digital light source projects each layer onto it. The light causes the resin to cure onto the platform, creating a solid sliver of the printed object. The light source can be a digital micromirror display (DMD) and a focusing lens. This light source is filtered so that only the necessary wavelengths are exposed to the resin, Layers are built on top of each other until each layer has completed, where the part is then removed from the build platform for post-processing. These printers accept digital (e.g., .STL or CAD) files that can be cut into layers in preparation for printing using a so-called “slicing” software. It is found in two main arrangements: DLP can print parts upsides down (known as bottom-up printing) or right-side-up (known as top-down printing).

Three-dimensional (3D) printing is carried out using a bioink formulation including a molecularly cleavable polymer. A bioink formulation, as used herein, refers to a formulation that can be used in 3D printing to provide a biocompatible 3D printed object that can include viable cells. The polymer precursors used in the bioink formulation should be photoactive, so that the polymer precursors can be polymerized upon exposure to light of the appropriate wavelength. The molecularly cleavable polymer allows the printed object to be partially degraded after printing in order to more closely resemble tissue, and in particular soft tissue.

The bioink formulation includes a mixture of a biocompatible cleavable polymer precursor, a biocompatible photoactive non-cleavable polymer precursor, and a photoinitiator. In some embodiments, the polymer precursors and the photoinitiator are stored separately and combined in the vat before use. Polymer precursors are the monomers or pre-polymers that react in order to form a polymer. Polymerization occurs through photoactivation of the polymer precursors.

Photoactivation is the activation of a polymer precursor by light. Photoactivation catalyzes polymerization of the polymer precursor. Typically a photoinitiator is included in the bioink to convert photolytic energy into a reactive species such as a radical or cation. Examples of photoinitiators include 12959, LAP, Eosin Y, and Ru/SPS, and others are known to those skilled in the art. See Bagheri A., and Jianyong J., ACS Appl. Polym. Mater. 2019, 1, 4, 593-611, the disclosure of which is incorporated by reference. The wavelength used will generally depend on the photoinitiator used. Examples of suitable wavelengths include from about 200 nm to about 500 nm, from about 250 nm to about 450 nm, or from about 300 nm to about 400 nm.

Because of their biocompatibility, preferred polymers for use in the bioink formulation are hydrogels, since hydrogels are water-rich polymers that can hold considerable amounts of water and are benign to embedded cells. Hydrogels are polymeric networks with hydrophilic chains crosslinked either covalently or physically (via intra- and intermolecular attractions). Biocompatible hydrogels include natural hydrogels and synthetic hydrogels. Examples of natural hydrogels include hyaluronic acid, hyaluronic acid derivatives (e.g., hyaluronic acid methacrylate), silk (e.g., silk fibroin or silk sericin), collagen, gelatin, gelatin derivatives (e.g., gelatin methacryloyl (GelMA)) and alginate. Examples of synthetic hydrogels include polyethylene glycol derivatives and polyvinyl alcohol derivatives.

The bioink formulation includes both a cleavable and non-cleavable polymer precursor. As used herein, cleavable and non-cleavable refer to the intended roles of the polymers formed from the precursors, and not an inherent property of the polymer precursor itself. Whether or not a polymer is cleavable in this context depends on the agent that is eventually used to cleave one of the polymers. For example, a hyaluronic acid derivative can be either a cleavable or non-cleavable polymer, depending on whether or not the polymer is subsequently contacted with a hyaluronidase (Hase), which is capable of cleaving the hyaluronic acid within the hyaluronic acid derivative.

In some embodiments, the non-cleavable polymer precursor is gelatin methacryloyl (GelMA). Sun et al., Polymers (Basel)., 10(11): 1290 (2018). A variety of different concentrations of GelMA can be used in the bioink formulation. In some embodiments, the GelMA has a concentration ranging from 1% to 10% by weight. In other embodiments, the GelMA has a concentration ranging from 2.5% to 7.5% by weight. In some embodiments, the GelMA has a concentration ranging from 1% to 5% by weight, while in other embodiments the GelMA has a concentration ranging from 5% to 9% by weight.

In some embodiments, the cleavable polymer precursor is a hyaluronic acid derivative. Hyaluronic acid derivatives are hyaluronic acid polymers that have been modified to include an additional chemical group. Examples of hyaluronic acid derivatives include hyaluronic acid alkyl derivatives. An example of a specific hyaluronic acid derivative is hyaluronic acid methacrylate (a.k.a. methacrylated hyaluronic acid). In some embodiments, the molecular weight (Mw) of the hyaluronic acid derivative ranges from about 10 kilodalton (kDa) to about 1,500 kDa. In further embodiments, the molecular weight of the hyaluronic acid derivative ranges from about 50 kDa to about 500 kDa, while in further embodiments the molecular weight of the hyaluronic acid ranges from about 75 kDa to about 125 kDa.

In some embodiments, the hyaluronic acid derivative is hyaluronic acid methacrylate (HAMA). Methods are known to those skilled in the art to tune the properties of methacrylated hyaluronic acid hydrogels for soft tissue engineering applications. Spearman et al., J Biomed Mater Res A, 108(2):279-291 (2020). One way to change the properties of the hydrogel used in the bioink formulation is to change its concentration. In some embodiments, the HAMA has a concentration ranging from 0.1% to 10% by weight, while in other embodiments the HAMA has a concentration ranging from 0.5% to 5% by weight, or from 0.75% to 3% by weight.

In some embodiments, the cleavable polymer precursor is HAMA, and the non-cleavable polymer precursor is GelMA. The concentrations of the two polymers in bioink formulations including these two polymer precursors can vary. In some embodiments, the GelMA has a concentration ranging from 1% to 7.5% by weight, and the HAMA has a concentration ranging from 1.25% to 3% by weight, in further embodiments GelMA has a concentration ranging from 2% to 6% by weight, and the HAMA has a concentration ranging from 1% to 2.5% by weight, while in a yet further embodiment the GelMA has a concentration ranging from 3% to 4% by weight and the HAMA has a concentration from 0.75% to 1.5% by weight.

As noted earlier, whether the polymers are considered cleavable or non-cleavable depends on the agent later used to cleave the polymer. In some embodiments, the non-cleavable polymer precursor is GelMA and the cleavable polymer precursor is HAMA. HAMA may be rendered cleavable by using the agent hyaluronidase, which cleaves HAMA, but which does not cleave GelMA. However, the roles of the polymers can be reversed by using a different agent to cleave the polymer. For example, if the degrading agent used is a matrix metalloproteinase (e.g., collagenase), and a HAMA/GelMA mix is used, the non-cleavable polymer is HAMA, and the cleavable polymer precursor is GelMA.

In some embodiments, the bioink formulation further comprises cells which will be embedded in the resulting hydrogel. Examples of suitable cells for use in the bioink formulation are described herein. In some embodiments, the bioink formulation comprises from about 1×10¹ to about 1×10⁹ viable cells, or from about 1×10² to about 1×10⁸ viable cells, or from about 1×10³ to about 1×10⁷ viable cells, or from about 1×10⁴ to about 1×10⁷ viable cells, or from about 1×10⁵ to about 1×10⁷ viable cells (all being cells per milliliter).

The bioink formulation can also include one or more additives. Non-limiting exemplary additives for the ink compositions include diluent synthetic polymers (e.g., polyethylene glycol, polypropylene glycol, poly(vinyl alcohol), poly(methacrylic acid)), drugs (e.g., antibiotics such as penicillin and streptomycin), cell nutrients (e.g., proteins, peptides, amino acids, vitamins, carbohydrates (e.g., starches, celluloses, glycogen), and minerals (e.g., calcium, magnesium, iron), synthetic or naturally occurring nucleic acids, absorbers to limit light penetration, inhibitors (e.g., scavengers and quenchers), refractive index modifiers (e.g., iodixanol), and nanocomposite components such as graphene or silica. The bioink formulation can comprise one or more additives in an amount of 0 wt % to about 25 wt % of the composition, based on total weight of the composition.

### Method of Three-Dimensional (3D) Bioprinting

Another aspect of the invention provides a method of three-dimensional (3D) bioprinting. The method can vary depending on the method of 3D printing being used. In the case of DLP printing, the method includes the steps of providing a biocompatible cleavable polymer precursor, a biocompatible non-cleavable polymer precursor, and a photoinitiator in a 3D bioprinter vat; repeatedly photoactivating the biocompatible polymer precursors in the 3D bioprinter vat on a build plate immersed in the vat to form a 3D bioprinted object comprising polymers having a series of predefined shapes across the vertical direction based on a set of sliced images; and treating the 3D bioprinted object with an agent that cleaves chemical bonds within the cleavable polymer.

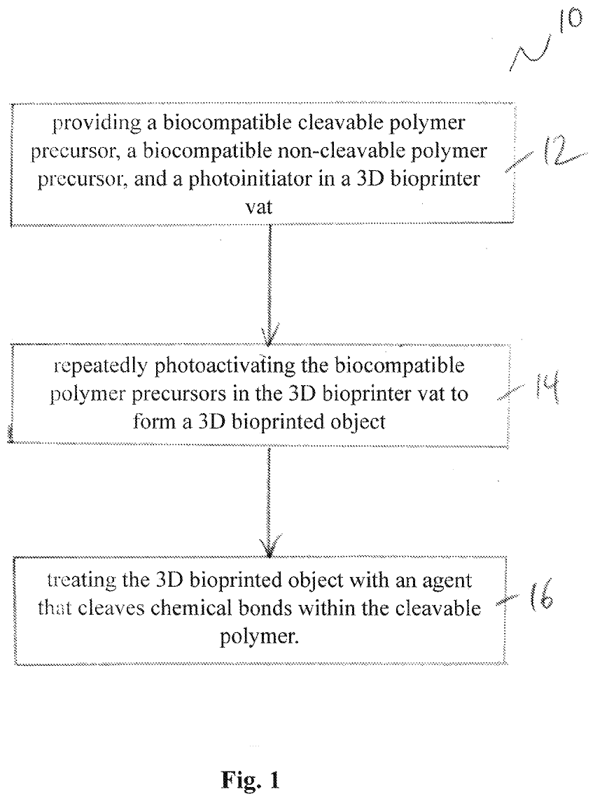

The steps of the method are shown in FIG. 1, which show a method of 3D printing 10 including the steps of providing a biocompatible cleavable polymer precursor, a biocompatible non-cleavable polymer precursor, and a photoinitiator in a 3D bioprinter vat 12, repeatedly photoactivating the biocompatible polymer precursors in the 3D bioprinter vat on a build plate immersed in the vat to form a 3D bioprinted object 14; and treating the 3D bioprinted object with an agent that cleaves chemical bonds within the cleavable polymer 16.

In other embodiments, the bioink formulation can be used with different types of 3D printing methods. In some embodiments, the bioink is applied by being extruded using an extrusion print-head. In other embodiments, the bioink is applied using a stereolithographic printing apparatus. The steps involved in carrying out these methods are known to those skilled in the art.

In some embodiments the biocompatible cleavable polymer precursor and the biocompatible non-cleavable polymer precursor are mixed in the vat. In other embodiments, the biocompatible cleavable polymer precursor and the biocompatible non-cleavable polymer precursor are kept separate and a method of 3D printing is used in which the cleavable and non-cleavable polymers are applied separately, providing an object in which specifically shaped regions of cleavable polymer can later be degraded.

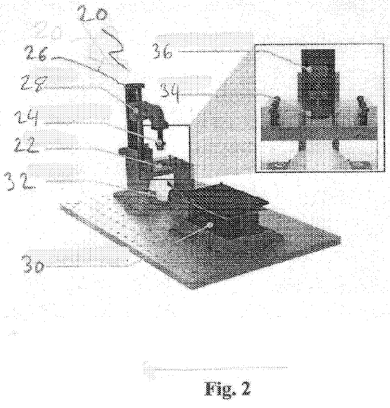

FIG. 2 provides a schematic illustration of a DLP-based 3D bioprinter developed by the inventors. The 3D printer 20 includes a vat 22 which is used to hold the polymer precursors, a build platform 24 upon which a 3D object is formed, an elevator 26 that can be used to raise and lower the build platform 24, and a motor assembly 28 that provides the force to move the build platform 24. The 3D printer 20 also includes a projection system 30 that creates light to shine upon the build platform 24 to photoactivate the polymer precursors, and a mirror 32 to angle the light from the projection system 30 so that it can reach the build platform 24. An expanded view of the vat 22 shows the bioink 34 contained within the vat 22, and a 3D bioprinted object 36 being formed upon the build platform.

The method includes the step of repeatedly photoactivating the biocompatible polymer precursors in the 3D bioprinter vat on a build plate immersed in the vat to form a 3D bioprinted object comprising a mix of polymers having a series of predefined shapes across the vertical direction based on a set of sliced images. Photoactivating the polymer precursors (or more specifically, the photoinitiator) causes the polymer precursors to polymerize, forming a predefined polymer shape on the build plate. The polymer precursors are photoactivated a number of times corresponding to the number of sliced images making up the software representation of the 3D printed object. Between photoactivations, the build plate is raised to allow additional polymer precursor to flow onto the build plate (and the incomplete 3D bioprinted object on the build plate) for the subsequent round of photoactivation. Once the 3D bioprinted object has been completed, it can be washed and/or further cured, as known by those skilled in the art.

In some embodiments, any one or more steps of the 3D printing method can be performed at a temperature from about 1° C. to about 99° C., or from about 10° C. to about 75° C., or from about 20° C. to about 50° C., or from about 25° C. to about 37° C. In some embodiments, all steps of the 3D printing method can be performed at a substantially constant temperature (e.g., no temperature change is required). Preferably, the 3D printing method is carried out at a temperature where photoactivation will result in fairly rapid polymerization of the polymer precursors, and benign to any cells that are present.

The mechanical properties of the 3D bioprinted object can be tuned based on the identity and concentrations of the polymers used. Other parameters, such as the concentration of the agent, and time of treatment, can also be used to tune the properties of the 3D bioprinted object.

After the 3D bioprinted object has been prepared, it is treated with an agent that cleaves chemical bonds within the cleavable polymer of the mix of polymers. For example, where a HAMA/GelMA mix is used, hyaluronidase can be used as the agent to break down hyaluronic acid within HAMA. Hyaluronidase is an endoglycosidase that breaks down hyaluronic acid into monosaccharides by cleaving its glycosidic bonds. Any agent (e.g., a chemical agent) that specifically cleaves one or more chemical bonds (i.e., molecular cleavage) in the cleavable polymer can be used. The agent used should provide specific cleavage in order to avoid degrading the non-cleavable polymer, which could result in a general degradation of the 3D bioprinted object. Typically, the 3D bioprinted object is treated with an agent by immersing the 3D bioprinted object in a solution including the agent. The concentration of the agent, and the time and temperature at it which it is used, vary depending on the particular agent. For example, when hyaluronidase is used to cleave HAMA, a 1,000 U mL⁻¹ solution of hyaluronidase at a temperature of about 37° C. for about 24 hours was shown to be effective. The time and concentration will vary depending on the activity of the enzyme being used. For example, collagenase was shown to be effective at a concentration of about 1 U mL⁻¹ for about 24 hours.

Because of their ability to specifically catalyze certain reactions, in some embodiments the agent is an enzyme. Enzymes are known to those skilled in the art that are capable of cleaving a wide variety of different substrates. For example, dextranase can be used to cleave dextran, alginate lyase can be used to cleave alginate, collagenase can be used to cleave collagen-based hydrogels, and protease XIV or α-chymotrypsin can be used to cleave silk fibroin or silk sericin. In some embodiments, the enzyme is a glyclosidase, which can be used to cleave bonds within a sugar molecule, or a protease, which can be used to cleave bonds within a protein. Since many enzymes involved in biochemical processes have evolved to be most effective at body temperature, they are typically most effective at a temperature from about 35° C. to about 40° C.

The polymer precursors, and the resulting polymers in the 3D bioprinted object, can be any of the polymers described herein. In some embodiments, the non-cleavable polymer is gelatin methacryloyl (GelMA), while in further embodiments the cleavable polymer is a hyaluronic acid derivative such as hyaluronic acid methacrylate (HAMA). In addition, the ratio of the two polymers can be used to vary the properties of the 3D bioprinted object, such as how much the 3D object will be degraded when exposed to the agent. The ratio of cleavable to non-cleavable polymer can vary from about 10:1, 8:1, 6:1, 4:1, 2:1, 1.5:1, 1:1, 1:1.5, 1:2, 1:4, 1:6, 1:8, or from about 1:10.

In some embodiments, the mixture of biocompatible polymer (i.e., the biocompatible cleavable polymer and the biocompatible non-cleavable polymer) further comprises cells. Standard cell culture techniques are typically used when handling the cells. In embodiments in which the 3D bioprinted object comprises cells, a portion of or the entire printed article can be placed under standard cell culture conditions (e.g., temperature, pressure, nutrient concentrations, etc.) in order for the cells to remain viable.

Viable cells that can be included in a 3D printed object include prokaryotic and eukaryotic cells. Non-limiting examples of eukaryotic cells include mammalian cells (e.g., stem cells, progenitor cells and differentiated cells). Stem cells have the ability to replicate through numerous population doublings (e.g., at least 60-80), in some cases essentially indefinitely, and also have the ability to differentiate into multiple cell types (e.g., pluripotent or multipotent). Other viable cells include immortalized cells that do not undergo normal replicative senescence, and can proliferate essentially indefinitely. Other living cells include embryonic stem cells, amniotic fluid stem cells, cartilage cells, bone cells, muscle cells, skin cells, pancreatic cells, kidney cells, nerve cells, liver cells, and the like. Viable cells are living cells.

### Three-Dimensional (3D) Objects

In some aspects of the invention, a three-dimensional (3D) object prepared from a bioink formulation is provided. The 3D object can be prepared by providing a bioink formulation comprising a mixture of a biocompatible cleavable polymer precursor, a biocompatible non-cleavable polymer precursor, and a photoinitiator in a 3D bioprinter vat; repeatedly photoactivating the bioink formulation in the 3D bioprinter vat on a build plate immersed in the vat to form a 3D bioprinted object comprising a mix of polymers having a series of predefined shapes across the vertical direction based on a set of sliced images; and treating the 3D bioprinted object with an agent that cleaves chemical bonds within the cleavable polymer of the mix of polymers.

The 3D object comprises two polymers (e.g., hydrogels); one that remains intact and one that has been cleaved using an agent. Suitable cleavable and non-cleavable polymers are described herein. In some embodiments, the cleavable polymer is hyaluronic acid methacrylate and the non-cleavable polymer is gelatin methacryloyl. For example, the 3D object can include GelMA having a concentration ranging from 2% to 6% by weight, and HAMA having a concentration ranging from 1% to 2.5% by weight. The amount of polymer obtained from the cleavable polymer precursors can be reduced depending on the amount of degradation that occurs. For example, the amount of polymer obtained from the cleavable polymer precursor present in the 3D object can be decreased by 10%, 20%, 30%, 40%, 50%, 60%, 70%, 80%, 90%, or even 100% in different embodiments of the invention. A 3D object prepared using a cleavable polymer can have a porous structure, and/or can include specific cavities or channels found in organs such as blood vessel channels.

The 3D objects prepared from the bioink formulations can have essentially any size and shape that can be obtained using a 3D printer. In some embodiments, the 3D object is an object intended for medical or pharmaceutical use, such as a tissue scaffold (e.g., an artificial transplant support), which may or may not include living cells. In some embodiments, the 3D object is a soft tissue construct (e.g., an artificial organ), which again may or may not include living cells. In some cases, the 3D object can be personalized for a specific subject by basing the 3D object on an image obtained from magnetic resonance imaging, computed tomography, or ultrasound. A wide variety of tissue engineering applications for 3D-printed hydrogels are known to those skilled in the art. Advincula et al., MRS Commun., 11(5):539-553 (2021).

In some embodiments, the 3D object comprises one or more embedded viable cells and/or cell types. In some embodiments, the printed 3D article is a scaffold for depositing and/or growing cellular tissue. A scaffold for cellular growth can have any suitable three-dimensional shape or dimensions. As a non-limiting example, a scaffold can comprise a stack of alternating layers of strands comprising the cleavable and non-cleavable polymers. When the polymers contain cells, the cells may be substantially uniformly distributed throughout the polymer, or they may be suspended within a part of the polymer.

In some embodiments, the 3D bioprinted object is a tissue construct. In particular, the 3D bioprinted object can be a soft tissue construct. Soft tissues connect and support other tissues and surround the organs in the body. They include muscles (e.g., the heart), fat, blood vessels, nerves, tendons, and tissues that surround the bones and joints. Examples of 3D soft tissue constructs include skin, musculoskeletal tissue, cardiac tissue, heart valve, liver, and neuronal tissue. The cells included in the tissue construct are preferably the type of cells normally found in the particular type of tissue, or precursor cells (e.g., stem cells) that will result in that particular type of tissue.

Accordingly, in some embodiments, the tissue construct is a liver tissue construct and the mix of polymers further comprises hepatocytes. In further embodiments, the tissue construct is a muscle tissue construct and the mix of polymers further comprises myoblasts. In yet further embodiments, the tissue construct is brain tissue construct and the mix of polymers further comprises neural progenitor cells.

The present invention is illustrated by the following example. It is to be understood that the particular example, materials, amounts, and procedures are to be interpreted broadly in accordance with the scope and spirit of the invention as set forth herein.

### EXAMPLES

### Example 1: Molecularly Cleavable Bioinks Facilitate High-Performance Digital Light Processing-Based Bioprinting of Functional Volumetric Soft Tissues

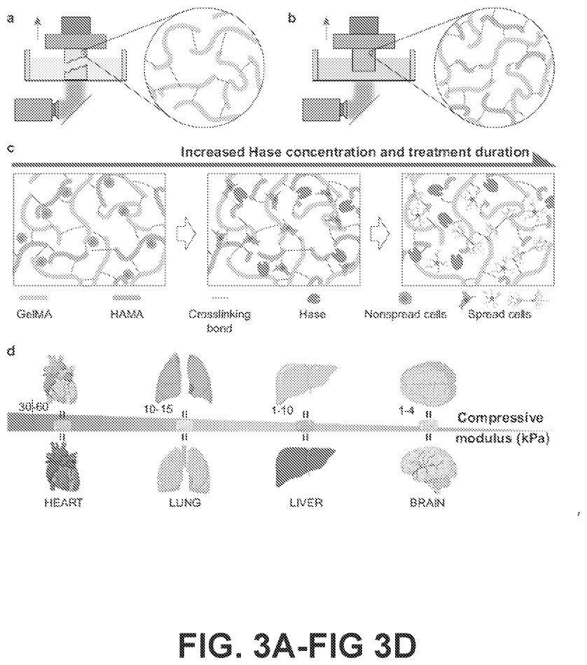

In this study, we attempt to address this unmet need of bioink development through a sufficiently simple yet highly efficient materials approach, via the use of target bioinks (e.g., gelatin methacryloyl (GelMA) as a demonstration) homogeneously mixed with hyaluronic acid (HA) methacrylate (HAMA), both photoactive, to enable DLP bioprinting with simultaneous structural fidelity and bioactivity (FIG. 3). This method generates good volumetric printability due to the high mechanical performance of the HAMA-mixed GelMA bioink during bioprinting. Of note, post-enzymatic digestion of the HAMA molecules using hyaluronidase (Hase) subsequently leads to final constructs with well-maintained structural fidelity. More importantly, desired mechanical properties within a wide range (above 100 kPa down to around 1 kPa), are precisely tunable by the enzymatic digestion process. A mechanical property library in relation to bioink formulations and digestion parameters is further established through mathematical modeling. We demonstrate the wide applicability of this approach by bioprinted multiple functional soft tissues from the muscles to of utmost excitement, the brain, the softest organ of the human body, Axpe, E. et al., Nat. Commun. 11, 3423 (2020), all in truly volumetric, structurally sophisticated architectures. This design strategy of mechanically tunable, molecularly cleavable bioinks presents an enabling means to customize the mechanical and biological properties of DLP-bioprinted tissue constructs towards creating not only biomimetic structures but also tissue-matching mechanical microenvironments for ultimate functionalities.

### Results and Discussion

### Printability of GelMA Bioinks Mixed by HAMA

GelMA, as a photocrosslinkable derivative of gelatin, possesses advantages of favorable cyto/biocompatibility, intrinsic bioactive motifs, and controllable on-demand gelation kinetics. Ying, G., et al., Bio-Des. Manuf 1, 215-224 (2018). GelMA-based bioinks, including hybrid bioinks of GelMA and HA-derivatives, have been widely used for extrusion-based bioprinting and tissue engineering applications, including those that are soft in nature thanks to the recently popularized embedded bioprinting strategy. Zhou, F. et al., Biomaterials 258, 120287 (2020). However, it is still challenging to obtain soft 3D hydrogel constructs of GelMA (or any other similar bioinks) via the DLP-based bioprinting technique, due to its weak mechanical properties at low concentrations, which are insufficient to support the gravity of layer-by-layer 3D-fabricated structures in particular in the bottom-up configuration. For example, the 10% (all expressions are in w/v % unless otherwise noted) GelMA was capable of being DLP-printed into a 3D cubic construct. In comparison, the reduced concentration of GelMA at 7.5% resulted in only partial printing, as evidenced by the break in the middle of the cube. As expected, the printing failed from the very first layer when the GelMA concentration was further decreased to 5.0%.

While it is possible, in principle, to produce relatively soft hydrogel constructs with the top-down DLP setup, it also does not support the (bio)printing of high-fidelity patterns due to the surface tension effect, in addition to the significant waste of bioink that has to fill the entire vat during the fabrication process. Li, W. et al., Adv. Healthc. Mater. 9, 2000156 (2020). Computed axial lithography (CAL) is another recent technique that allows efficient decoupling of mechanical performances of bioinks and bioprinting shape fidelity. Kelly, B. E. et al., Science 363, 1075-1079 (2019). However, its build volume can be limited due to the need for light penetration through the entire bioink tank (many bioinks/photoinitiators are absorption-bearing). In comparison, DLP-based (bio)printing enables fabrication of large-volume constructs due to its layer-by-layer photocrosslinking nature. Anandakrishnan, N. et al. Adv. Healthc. Mater. 10, 2002103 (2021).

We formulated a collection of GelMA/HAMA inks with different concentrations as well as molecular weights (M_{w}) of HAMA (10-1500 kDa), and explored their volumetric printability using DLP. The HAMA and GelMA used in this study were analyzed by ¹H nuclear magnetic resonance spectra (NMR), to confirm successful modification.

Specifically, the degree of methacryloyl-substitution for GelMA was determined at 81.7±0.7%. In addition, the primary amine contents were measured by the trinitrobenzene sulfonic acid (TNBSA) assay, which for gelatin and GelMA were 8.1±0.2 μmol mg⁻¹ and 1.5±0.1 μmol mg⁻¹, respectively.

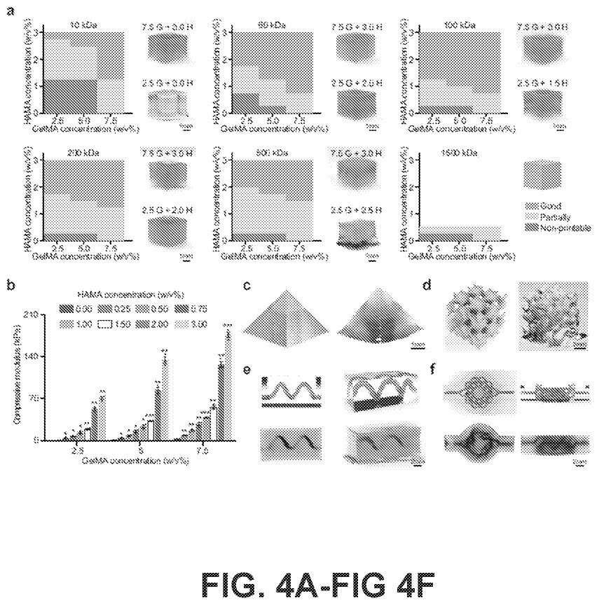

A cube with a size of 4×4×4 mm³ was printed by each formulation of the inks with a 100-μm layer thickness and a 30-s crosslinking time for each layer. The printability maps that we generated accordingly indicated that the printable areas (i.e., number of green squares in each map) were in general enlarged when HAMA concentrations were increased, at the M_{w} under 500 kDa (FIG. 4a). Among these, printability of the inks containing HAMA of M_{w}=10 kDa and M_{w}=500 kDa exhibited limited areas on the maps because of their weak mechanical properties and high viscosity values, respectively. Moreover, the inks containing HAMA (M_{w}=1500 kDa) displayed completely non-printable features due to their overly high viscosities. On the contrary, the largest green printable areas in this set of printability maps represented HAMA of M_{w}, =100 kDa, endowing better printability compared with the inks containing 60-kDa or 200-kDa HAMA. A similar trend of printability variations was observed when we attempted to increase or decrease the GelMA concentrations in the inks as well. Therefore, an optimal HAMA M_{w} of 100 kDa was selected for the subsequent experiments, allowing successful 3D printing of the inks covering 2.5% GelMA homogeneously mixed with HAMA from 1.25 to 3.00%, 5.0% GelMA with HAMA from 1.00 to 3.00%, and 7.5% GelMA with HAMA from 0.75 to 3.00%. As noted above, GelMA inks at these concentrations (2.5-7.5%) by themselves, are not directly printable with the DLP method unless mixed with other photocrosslinkable components (e.g., poly(ethylene glycol)-diacrylate (PEGDA) Grigoryan, B. et al., Science 364, 458-464 (2019), or methacrylated poly(vinyl alcohol) (PVA-MA) Lim, K. S. et al., Biofabrication 10, 034101 (2018)) again resulting in stiffer hydrogels. Though, even printable in the presence of these additives, the activities of embedded cells would likely become strongly affected by the densely crosslinked polymer networks.

The compressive moduli of the constructs fabricated with GelMA/HAMA (HAMA M_{w}=100 kDa) inks were further evaluated. As shown in FIG. 4b, the mechanical properties were augmented with the aid of inclusion of HAMA molecules. Taking 5.0% GelMA as an example, the compressive moduli improved from 1.3±0.2 to 134.4±3.1 kPa when the HAMA concentration was increased from 0.0 to 3.0%. Similar tendencies were observed for 2.5% GelMA and 7.5% GelMA. These results of mechanical properties were in accordance with what we obtained from the printability maps, where the formulated inks featuring compressive moduli>20 kPa generally exhibited good printability in the DLP-based method.

We subsequently investigated the printing parameters for the ink made of GelMA/HAMA (5.0%/3.0%), which was the representative ink formulation with good printability. We explored the influence of photoabsorber and exposure time on the curing depth of the ink (GelMA/HAMA, 5.0%/3.0%). In DLP (bio)printing, photoabsorber is oftentimes used to attenuate excessive light and adjust the photopolymerization kinetics to achieve the desired layer thickness, benefiting the printing fidelity. Huh, J. et al., Biofabrication 13, 034103 (2021). Ponceau 4R was chosen as the photoabsorber in our visible light-based bioprinting system, since its absorbance spectrum is identified to encompass the visible-light wavelengths. Sayar, S. & Ozdemir, Y., Food Chem. 61, 367-372 (1998). The successful control over the optical penetration length by addition of Ponceau 4R and its cytocompatibility have been proven in our previous studies. Wang, M. et al., Adv. Mater. 34, e2107038 (2022). The working curve represents the photopolymerization kinetic of the ink and serves as a quick estimation of printing settings. We plotted the working curves of the inks made of GelMA/HAMA (5.0%/3.0%) via Eq. (1):

$${C}_{d} = {D}_{p} ln \frac{E}{{E}_{c}}$$

where C_{d} is the cure depth, Dₚ is the light penetration depth, E is the irradiation dosage, and E, is the energy required for achieving the gelation point. The irradiation dosage was calculated from light intensity multiplied by exposure time, and the curing depth directly measured from the optical microscopy image of the printed construct. For the ink of GelMA/HAMA (5.0%/3.0%) without photoabsorber, the curing depths ranged from 576.0±32.7 to 1162.3±67.0 μm when the crosslinking times were changed from 5 to 30 s. By adding the photoabsorber, the curing depths could be gradually adjusted to 104.7±12.3 to 633.3±24.3 μm (1% Ponceau 4R), 34.7±3.7 to 354.0±16.1 μm (2% Ponceau 4R), and 21.7±3.9 to 184.0±11.3 μm (3% Ponceau 4R). Considering the thickness demands of our printing were 50 to 300 μm, we chose 2% Ponceau 4R for further investigations.

Moreover, a radial pattern was utilized to assess the resolution at different crosslinking times with or without photoabsorber (2.0% Ponceau 4R) addition. The diameters of the central circles were raised when the exposure time was extended from 5 to 30 s, likely due to the fast diffusion of the free radicals, which was conversely reduced in the presence of photoabsorber to the inks. Meanwhile, the calculated resolution, which implied the smallest distance that could be distinguished between two independent points, was enhanced after incorporating the photoabsorber. The influence of exposure time on the resolution of the printed patterns was not as significant in the inks containing the photoabsorber compared to those without. Among all the parameters we evaluated, the radial diameter of 1.1 mm and the resolution of 200 μm, which were closest to our designed pattern, could be obtained using 15 s of exposure time and 2.0% of photoabsorber concentration.

By utilizing the optimized ink formulation and printing conditions (100-μm thickness and 15-s exposure time for each layer), we printed several representative 3D constructs featuring complex internal and external architectures. It was observed that a solid pyramid with clear sharp edges could be printed (FIG. 4c). Additionally, a gyroid featuring highly curved surfaces and interconnected internal pores was printed to demonstrate the ability to generate 3D porous scaffolds (FIG. 4d). We also confirmed the capacity to print 3D constructs having complex embedded channels, including the structure with a spiral channel (diameter=1 mm, FIG. 4e) and the structure containing a torus channel entangled with another torus knot-like channel (FIG. 4f). Grigoryan, B. et al., Science 364, 458-464 (2019). Perfusions with a color dye verified the pattern fidelities and the channel patency. These 3D geometries encompassing challenging features (sharp edges, interconnected pores, and sophisticated hollow channels) are highly desirable in tissue biofabrication applications, exhibiting again the printability of our GelMA inks homogeneously mixed by HAMA.

### Enzymic Digestion of HAMA and Cytocompatibility Assays

Hase can catalyze the hydrolysis of the β-1,4-glycosidic bond between hyaluronic acid monomers. Kadokawa, J., Chem. Rev. 111, 4308-4345 (2011). The concentration and duration of Hase treatments were anticipated to play primary roles in the precisely tunable mechanical properties of the DLP-printed constructs. The mechanical properties of GelMA/HAMA constructs decreased when the Hase concentration or digestion time was increased. This phenomenon was most clearly identified with GelMA/HAMA at 7.5%/1.5%, which endorsed a relatively high initial stiffness (56.6±2.3 kPa) that was significantly reduced to 10.1±0.6 kPa after enzymatic digestion to a value slightly>7.5% pure GelMA (3.0±0.5 kPa). It should be pointed out once more that the 7.5% GelMA by itself is almost non-printable through the DLP method. More importantly, a wide range of compressive moduli could be achieved through the digestible HAMA network, from ˜180 kPa all the way down to 1 kPa, which is suitable for modeling multiple soft tissues including but not limited to the brain (1-4 kPa), the liver (1-10 kPa), the lung (10-15 kPa), and the heart (30-60 kPa). Guimaraes, C. F. et al., Nat. Rev. Matier. 5, 351-370 (2020).

Fluorescein isothiocyanate (FITC)-conjugated HAMA-mixed GelMA was used to further confirm the enzymatic cleavage of HAMA. Microscopic images presented clear digestion signs under different Hase concentrations after 24 h of incubation. The decreased fluorescence intensity suggested that a rapid digestion occurred under a higher Hase concentration and a lower concentration of GelMA. For instance, the disc printed with 2.5% GelMA mixed with 1.5% HAMA displayed a near-complete digestion after 24 h of Hase treatment at the concentration of 1000 U mL⁻¹. Another digestion analysis used consistent Hase concentration (1000 U mL⁻¹) but varied treatment durations. The results indicated that the digestion time also played a key role in HAMA cleavage, which coincided with the mechanical test results as well. In the process of Hase digestion, the cleavage of HAMA generated smaller pieces of disaccharide chains that were leached out. This was the possible reason that some of the labeled-HAMA was observed to diffuse out with digestion. Therefore, the crosslinked HAMA content was reduced, and the overall crosslinking density was decreased, leading to the lowered and tunable mechanical stiffness of the printed constructs following Hase treatment.

Further investigations regarding Hase diffusion within the GelMA/HAMA (2.5%/1.5%) hydrogels exhibited that FITC-dextran (M_{w}=60 kDa), the replacement of Hase (M_{w}=55 kDa) for easier visualization, had an effective diffusion coefficient of 3.5×10⁻⁸ cm² s-1, as calculated by tracking the axial diffusion. In reality, however, with the Hase digestion undergoing, its diffusion rate in the GelMA/HAMA hydrogel would be dynamic since the digested hydrogel network could also influence the overall diffusion. Indeed, when we conducted the same diffusion test using the Hase-treated (1000 U mL⁻¹ for 24 h) GelMA/HAMA samples as an example, we did observe a faster FITC-dextran diffusion speed compared with the untreated control. The effective diffusion coefficient was also calculated to elevate to 1.8×10⁻⁷ cm² s⁻¹ in the Hase-digested GelMA/HAMA hydrogels. To this end, once knowing such diffusional parameters in different relevant setups, it would be convenient to precisely design the conditions that would allow to achieve uniform diffusion of Hase and digestion.

To fully understand the influence of ink formulation and Hase administration, a mathematical model was established to predict the compressive moduli of the digested constructs based on experimental results, using Eq. (2) expressed below:

$$Compressive modulus ( kPa )$$

$$= - 11 \; 3.3016 + 5.8 \; 599 GelMA - 0.1708 Digestion time - 0.0173 Hase$$

$$+ 66. 1 \; 107 HAMA - 0.3 \; 595 GelMA \times Digestion time$$

$$- 0. 099 GelMA \times Hase + 6.9 \; 941 GelMA \times HAMA$$

$$- 2.1007 Digestion time \times HAMA - 0.0 \; 458 Hase \times HAMA$$

$$+ 0.1 \; 663 Digestion {time}^{2} + 0.0001 {Hase}^{2}$$

The equation illustrates that the HAMA concentration was the most significant factor in determining the moduli with a coefficient of 66.11 kPa×% HAMA⁻¹. This was followed by the product of GelMA and HAMA (6.9941% GelMA⁻¹×% HAMA⁻¹) and then by the GelMA concentration (5.8599% GelMA⁻¹). However, it should be noted that, this equation should not be used to determine the relative impact of each factor because the coefficients were scaled to accommodate the units of each factor and the intercept was not at the center of the designed space. To determine the relative impact of each term, the model equation in terms of coded values is given below in Eq. (3), which was able to make predictions regarding the responses for given coded levels of each factor.

$$Compressive modulus ( kPa )$$

$$= 28. 76 + 3 0.87 A - 3 2.44 B - 31.7 C + 39.73 C - 1 0.79 A \times B - 12.33 A \times C$$

$$+ 13.11 A \times D - 18.91 B \times D - 1 7.18 C \times D + 2 3.95 {B}^{2} + 2 6.58 {C}^{2}$$

where A is the concentration of GelMA (%), B is the digestion time of Hase (h), C is the concentration of Hase (U mL⁻¹), and D is the concentration of HAMA (%). By default, the high levels of the factors were coded as +1 and the low levels were coded as −1. The actual values were then scaled using this range. The coded equation was useful for identifying the relative impacts of the factors by comparing the factor coefficients. The statistical significances of the fitted second-degree polynomial model were assessed by the P-value of analysis of variance (ANOVA).

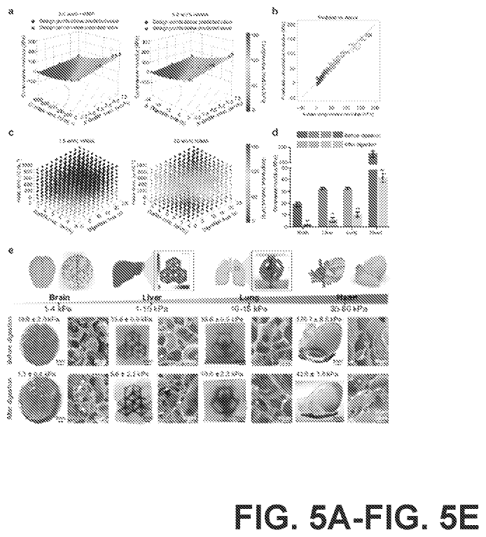

From the results, the coded HAMA concentration term displayed the highest impact on controlling the modulus value followed by digestion time, Hase concentration, and GelMA concentration. Surface plots of the model were determined by setting the Hase concentration to 1000 U mL⁻¹ and the HAMA concentrations to 1.5% or 3% (FIG. 5a). Analyses of the fit statistics revealed that the model had a coefficient of determination R² equal to 0.9590 and an adequate precision value of 78.28, suggesting an excellent fit and thus could be used to navigate the design space. FIG. 5b revealed the graph of the predicted values versus experimental values, whereby the closer a particular point was to the diagonal line, the better of the prediction. This plot indicated that prediction using this model was better at relatively low values of modulus (<130 kPa) than at higher modulus values (>130 kPa). FIG. 5c elucidated the establishment of parameter maps, by which any targeted moduli of mimic tissues could be navigated to find the initial concentrations of GelMA/HAMA to print, as well as the conditions of Hase concentration and digestion times for post-printing treatment, suggesting a good potential for multiple soft-tissue designs and fabrications. We further conducted additional verification experimental trials according to the predicted outcomes based on this established mathematical model. Target modulus values of 5, 15, 30, 65, and 110 kPa were chosen and the parameters used were calculated using numerical optimizations. This broad modulus range roughly covered our desired tissue moduli. The prediction interval was calculated beforehand to estimate an interval in which the mean of the additional trials would fall, at a probability of 95%. All five experimental moduli were found within the range of the respective prediction intervals (PIs), verifying the success of this simulation model.

Considering that this mathematical modeling approach provided distinct information of bioink formulation and digestion conditions of any target mechanical property, we selected three bioink compositions from this simulation to fabricate desired tissue-mimics, including the brain (GelMA/HAMA, 2.5%/1.5%), the liver or the lung (GelMA/HAMA, 5%/1.5%), and the heart (GelMA/HAMA, 7.5%/3.0%). As shown in FIG. 5d, the mechanical properties of the typical soft tissues in the human body, such as the brain, liver, lung, and heart, were recapitulated through different bioink formulations and their digestion parameters determined from the mathematical model. In these examples, the compressive moduli of the brain-mimic changed from 19.9±2.0 kPa (immediately post-fabrication) to 1.3±0.4 kPa (post-digestion of HAMA), liver-mimic from 33.6±0.9 kPa to 5.6±2.2 kPa, lung-mimic from 33.6±0.9 kPa to 10.6±2.3 kPa, and heart-mimic from 176.7±8.5 kPa to 42.9±3.8 kPa. All the mechanical properties of printed tissue-mimics were widely tuned following this procedure and achieved proper range of target stiffness. On the basis of these results, we concluded that this strategy could be used to precisely tune the mechanical properties of DLP-printed constructs through enzymatic digestion within acceptable prediction intervals.

In addition, we successfully mimicked various exemplary complex shapes (brain and heart) as well as the representative vascular patterns (liver and lung) with these constructs using DLP printing (FIG. 5e). The printed brain-like structure displayed clearly visible cerebral sulci and grooves. The heart-like construct featured the 3D anatomical shape containing the aorta and the left and right ventricles. Excellent printability and perfusable performances were also observed in the designs of the liver and the lung, whereby the truly volumetric hexagonal lobule units and the bioinspired alveolar structure each contained an embedded 3D anatomical vascular network. Of particular note, all the printed fine features were retained after enzymatic digestions, where the diameter of the embedded microchannels such as those in the hepatic lobule, the alveolar lung, and the cuboid became slightly enlarged post-digestion (FIG. 5e). Interestingly, the scanning electron microscopy (SEM) results revealed similar microstructures, with only slight difference of ultrasmall porosity profiles as observed in the freeze-dried printed constructs before and after enzymatic digestion (FIG. 5e). Note that SEM images do not directly reveal the interchain distances due to the possible packing of polymer chains during the freeze-drying process. Further porosity measurements via a nitrogen-adsorption method determined that the specific surface area of the Hase-digested samples increased when compared with the untreated controls, which was resulted from the additional pores generated after cleaving the HA chains. However, the average pore size was slightly reduced after Hase digestion, suggesting that the newly formed pores by molecular cleavage were indeed, small in scale that caused such a reduction in the average size of all the pores present in the system, to a good extent consistent with SEM observations that microscale porosities remained largely unchanged. Our findings led us to conclude that the GelMA/HAMA bioinks with highly tunable stiffness values (˜1-180 kPa demonstrated but likely beyond) yielded by post-printing enzymatic digestion, enable facile DLP printing of structurally sophisticated constructs while allowing recapitulation of soft-organ mechanical properties otherwise previously almost impossible.

We have now introduced the concept of molecularly cleavable bioinks as a methodology for 3D soft-tissue bioprinting and studied the possibilities with a representative collection of bioinks based on GelMA/HAMA, followed by the treatment with an enzyme specifically digesting the HAMA component. This method was further expanded to GelMA-cleavable GelMA/HAMA bioinks through digesting GelMA chains using collagenase. Greene T. et al., J. Appl. Polym. Sci., 134(11), 44585 (2017). Bioinks of GelMA/HAMA (2.5%/1.5%, M_{w} of HAMA=100 kDa) and GelMA/HAMA (2.5%/2.0%%, M_{w} of HAMA=100 kDa) were taken as examples to demonstrate the feasibility of digesting GelMA with collagenase and obtaining softer, HAMA-rich hydrogels. The compressive moduli of the GelMA/HAMA (2.5%/1.5%) constructs were decreased from 26.2±4.2, to 8.2±0.6 and 4.7±1.1 kPa by the treatment of 1- and 2-U mL⁻¹ collagenase, respectively, for 24 h. The same tendency could be found when the HAMA concentration was increased to 2.0%, while the impact on lowering hydrogel stiffness was not as significant as the GelMA/HAMA (2.5%/1.5%) constructs. Therefore, the alternative method of cleaving GelMA molecules from GelMA/HAMA-printed constructs also provided an option for lowering hydrogel stiffness through post-printing digestion. Other polymers, which can provide sufficient mechanical properties for bioprinting and be molecularly cleaved by specific enzymes, are possible options to serve as molecularly cleavable bioinks as well. The examples include but are not limited to photocrosslinkable moiety-functionalized polysaccharides, such as dextran and chitosan, as well as glycosaminoglycans, such as chondroitin sulfate and heparin.

### Tissue-Fabrication and Evaluations

As discussed, our unique strategy of post-bioprinting enzymatic digestion is motivated by the oftentimes contradicting requirements of both structural complexity and biological properties of these fabricated tissue-mimics, such as supporting the growth of cells originated from soft tissues to ensure proper functions. This is particularly problematic in the conventional DLP bioprinting method, which allows bioprinting of sophisticated patterns but only when the mechanics of the hydrogels are sufficiently high. Grigoryan, B. et al., Science 364, 458-464 (2019). To demonstrate the feasibility of our strategy in soft-tissue bioprinting using DLP, we first sought to evaluate the influence of the Hase enzyme on behaviors of multiple cell types. The results revealed that high enzyme concentrations and long treatment times had negative impacts on the survival and metabolic activities of NIH/3T3 fibroblasts cultured on the surfaces of well-plates. For this reason, NIH/3T3 fibroblasts were treated with lower enzyme concentrations (below 1000 U mL⁻¹) for 24 h. Neither cell survival nor metabolic activities were significantly affected under these conditions.

We further assessed the C2C12 myoblast behaviors within the GelMA/HAMA constructs after 1-day digestion by different Hase concentrations. An interesting finding was that the C2C12 myoblasts started to spread on day 3 of culture after 500 U mL⁻¹ of enzymatic digestion, which was in line with the cell morphology observed in GelMA (7.5%)-only control. Of note, the latter samples were fabricated through casing since GelMA alone at this concentration was not printable in 3D. Cell spreading within the GelMA/HAMA constructs was particularly attractive as the enzyme concentration was increased to 1000 U mL⁻¹. In these constructs, adding HAMA to GelMA followed by Hase digestion showed no negative effect on cell spreading. Hase cleaved interconnected HAMA chains, and thus provided more space for cell growth and spreading.

Considering that the encapsulated cells might leach out during HAMA digestion, we next explored the cell motility using the NIH/3T3 fibroblasts bioprinted in GelMA/HAMA constructs. While a small population of cells (16.0±2.2%) did leach out when the bioprinted samples were treated with Hase (1000 U mL⁻¹) for 24 h, the metabolic activities of the cells within the Hase-treated constructs over the subsequent culture period dramatically increased compared to the untreated controls. On the basis of cell morphological enhancements after enzymatic digestion, we then illuminated the great potential of the DLP-bioprinted GelMA/HAMA constructs followed by enzymatic digestion in the fabrication of biologically relevant tissue-mimics.

### Bioprinting of Skeletal Muscle Tissue

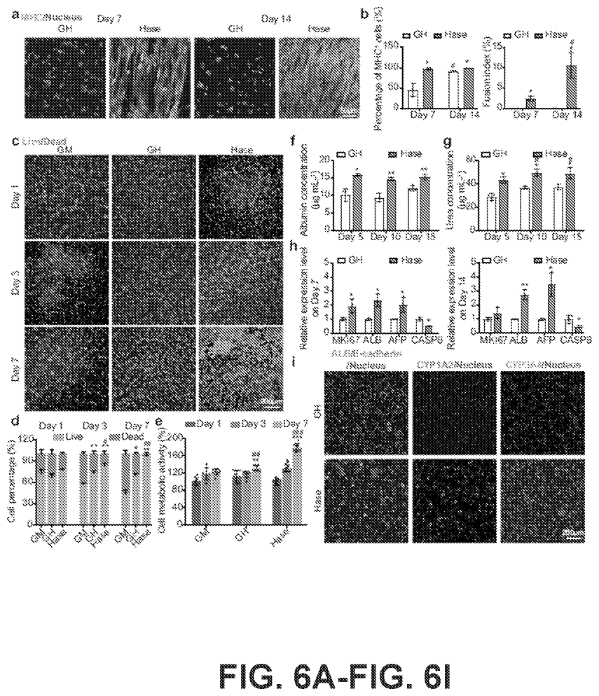

The first tissue we mimicked was the skeletal muscle, where we bioprinted C2C12 myoblast-embedded GelMA/HAMA (GH group, 7.5%/1.5%) bioink, and further digested the bioprinted muscular tissue in Hase at the concentration of 1000 U mL⁻¹ (Hase group) for 24 h. The C2C12 myoblasts cultured in the GH group and the Hase group were evaluated by immunofluorescence staining of myosin skeletal heavy chain (MHC), which is a myogenic differentiation marker. Lee, E. J. et al., Cell Death Dis. 9, 1092 (2018). As shown in FIG. 6a, b, the cells displayed an isolated growth pattern with limited cell spreading, as well as impaired myogenic biomarker expression in the GH group after both 7 days and 14 days of differentiation (day 10 and day 17 after Hase digestion). By contrast, the same cells in the Hase-digested samples started to fuse with surrounding cells and self-organized to form myotubes that exhibited spontaneous alignments in most if not all the regions (FIG. 6a, b). Cell fusion and myotube-formation became more significant after 14 days of differentiation, as also quantified by the fusion index plot. From the results we obtained, it was clear that the Hase-digested hydrogels effectively supported C2C12 cell spreading and this cleaved microenvironment favored the differentiation of the cells.

Myogenesis is a process of myoblast differentiation from single cells into multinucleated muscle fibers. Le Grand, F. & Rudnicki, M. A. Curr. Opin. Cell Biol. 19, 628-633 (2007). During this process, myoblasts recast spatial cellular arrangement over distances without a central coordinator to become well-ordered and multinucleated myotubes from a disordered state of individual, undifferentiated cells. Many rationales behind this process are still unclear; however, researchers have identified several physical factors to play an important role in guiding myoblasts self-organization. For example, it was revealed that C2C12 myoblasts spontaneously assembled into highly aligned myotube tissues when cultured on sufficiently soft yet fully isotropic gelatin substrates. Jensen, J. H. et al., Sci. Rep. 10, 13305 (2020). In our study, the enzymatic digestion process resulted in softer hydrogels matching the muscle tissue stiffness. The formed myotubes were likely influenced by the proper hydrogel stiffness and were observed to align only in the Hase-digested samples, which were consistent with the observations from the literature. Jensen, J. H. et al., Sci. Rep. 10, 13305 (2020).

### Bioprinting of Hepatic Tissue

To create a hepatic tissue model that mimics the native mechanical properties and cellular functions, we encapsulated hepatocyte-like HepG2/C3A cells into the bioprinted GelMA/HAMA (5%/1.5%) constructs followed by post-bioprinting enzymatic digestion (1000 U mL⁻¹ for 24 h, Hase group). We specifically compared the culture of HepG2/C3A cells within the Hase group to that before digestion (GH group), and 10% GelMA alone (GM group). The hydrogels made by 5% GelMA have been commonly adopted as the scaffolds for liver tissue engineering. Maharjan, S. et al., Bio-Des. Manuf 4, 157-170 (2021). Hence, we also obtained the 5% GelMA-alone group encapsulating HepG2/C3A cells through the casting method instead of DLP bioprinting, since the bioprinting failed when the GelMA concentration was decreased to under 10%. The live/dead assay illustrated that an obviously increasing level of live cells in the Hase-digested GelMA/HAMA constructs was observed across the 7 days of culture, whereas a constantly decreasing number of live cells could be found in the GM samples at days 3 and 7 (FIG. 6c, d). HepG2/C3A cellular aggregation in the Hase group started at day 3 after enzymatic digestion, with increasing aggregation size over the entire culture period. At day 7, many contiguous cellular aggregates merged together. The results were consistent with those of the cast 5% GelMA group, where the cellular aggregation enlarged throughout the same culture period. The cells in the Hase group were still highly viable and spread well even when cultured for 14 days, suggesting the enhanced cytocompatibility of our post-bioprinting digestion method. On the contrary, the live HepG2/C3A cells in the GH group exhibited slight increase over 7 days of culture, and only a few small aggregates were formed by the cells cultured in the GM and GH constructs (FIG. 6c, d). A similar trend was observed when the cells were cultured for up to 14 days. Metabolic activity assays demonstrated similar cellular responses (FIG. 6e) with the results from the live/dead assay. Hase-digested and 5% GelMA-cast structures presented excellent cell proliferations from 1 to 7 days; however, cells cultured in the GM and GH groups did not significantly improve over the period. These outcomes were consistent with the observations from previous studies, where cells cultured in GelMA at a higher concentration (10%) generally show limited activities. Li, W. et al., Adv. Healthc. Mater. 9, 2000156 (2020).

The secretions of albumin (ALB) and urea, which are important indicators of hepatic functions, Ma, X. et al., Biomaterials 185, 310-321 (2018), were further illustrated at higher productions by the encapsulated HepG2/C3A cells in the Hase group, when compared to the GH samples at the 5-day, 10-day, and 15-day time points (FIG. 6f, g). This observation implied the dramatic influence on functional maintenance of hepatic homeostasis with post-treatment of Hase. To further verify this hypothesis, the related gene expressions of cell proliferation markers (MKI67), liver function markers (ALB and alpha fetoprotein (AFP)), and apoptosis markers (caspase 8, CASP8) were investigated at days 7 and 14 of culture after Hase digestion. Significant upregulations of the MKI67, ALB, AFP genes along with a lower expression of the apoptosis marker CASP8 were found in HepG2/C3A cells cultured in the Hase group at both time points (FIG. 6h). Confocal images of the cells cultured in samples showed positive staining for both ALB and E-cadherin (FIG. 6i). Nevertheless, more than 2-fold and 6-fold increases of ALB and E-cadherin expressions, respectively, were recorded in the Hase-digested constructs, confirming that post-bioprinting digestion was of paramount importance in terms of supporting ALB-production and epithelial cell junction-formation of these hepatic cells. Encouraged by the observed functional enhancements, we proceeded to evaluate the cytochrome P450 (CYP) enzymes, which are a key enzyme family in liver metabolism defining detoxification and bioactivation processes. Lewis, P. L. et al., Acta Biomaler. 69, 63-70 (2018). As FIG. 6i indicated, both CYP1A2 and CYP3A4 presented higher expressions in the Hase group compared to the GH group, which displayed potentially enhanced the capabilities of metabolism and drug clearance. It is also worthwhile to mention that the higher CYP3A4 expression itself represents a maturation towards adult hepatocytes as fetal hepatocytes express a relatively low level of CYP3A4. Ma, X. et al., Proct Natl Acad. Sci. USA 113, 2206-2211 (2016).

### Bioprinting of Functional Mini-Brain Tissues

The brain is the most complex yet softest organ in the body. Lacour, S. P. et al., Nat. Rev. Mater. 1, 1-14 (2016). Recapitulating the native physicochemical microenvironment in support of neurons and glial cells in vitro is of paramount importance for their proliferation, differentiation, and functionalities. Liu, Z. et al., Adv. Mater. 30, 1705388 (2018). Nevertheless, the fabrication of low-stiffness, truly volumetric brain microtissues using 3D bioprinting has not yet been effectively realized, again because of the contradicting requirements of the ultra-low mechanical properties of bioinks and the structural fidelity, which is a major obstacle in conventional DLP bioprinting. Having demonstrated the ability to bioprint sophisticated 3D structures using our GelMA/HAMA bioink and to mimic the mechanical properties of soft tissues by subsequently digesting the HAMA component, we next proceeded to study the feasibility of this unique strategy in bioprinting ultrasoft tissues such as the brain and supporting the behaviors of encapsulated neural progenitor cells (NPCs).

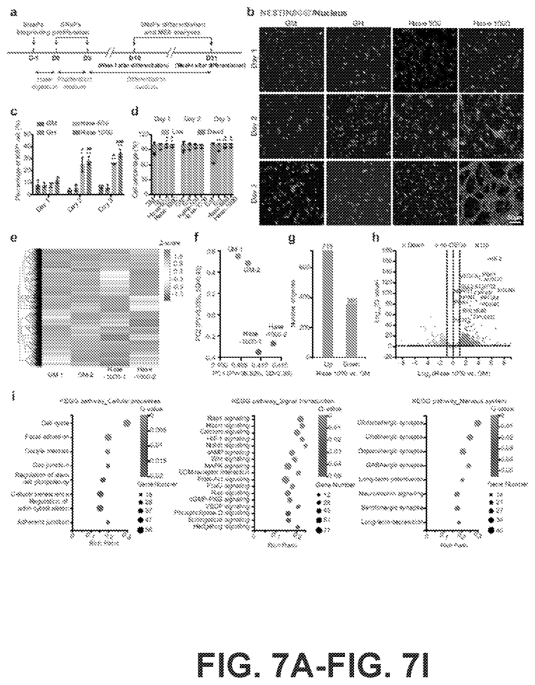

To achieve a robust and rapid generation of neuronal cells, we used human stem cell-derived neural progenitors as the seeding cell for bioprinting and subsequently differentiated them into neuronal cells post-bioprinting. Transcription factor-programming with the lineage-specific transcription factors emerges as a strategy for the induction of stem cell differentiation. Mertens, J. et al., Nat. Rev. Neurosci. 17, 424-437 (2016). Neurogenin-2 (NGN2), a transcription factor related to neuronal development, has been reported to produce Stem cell-derived NGN2-accelerated Progenitor cells (SNaPs) from human stem cell-derived NPCs. Wells M. F. et al., bioRxiv 116, 9527-9532 (2018). The generated SNaPs are multipotent cells, which are capable of differentiating into glia and neurons. The DLP-bioprinted SNaPs encapsulated in 10% GelMA (GM group), 2.5%/1.5% GelMA/HAMA (GH group), and 2.5%/1.5% GelMA/HAMA with Hase digestion (500 U mL⁻¹ for 24 h, Hase-500 group; or 1000 U mL⁻¹ for 24 h, Hase-1000 group), in the shape shown in FIG. 5e, were cultured in the proliferation medium for 3 days after Hase digestion, followed by neuronal induction in the differentiation medium (FIG. 7a). First, we investigated whether the Hase digestion of the GelMA/HAMA constructs influenced the proliferation and survival of SNaPs. Compared with the control groups of GM and GH, we found that the proliferation of SNaPs, indicated by the percentage of Ki67⁺ cells, increased significantly in the groups treated with Hase (Hase-500 and Hase-1000) at day 2 and day 3 after digestion (FIG. 7b, c). Additionally, the live/dead staining was conducted to evaluate SNaP viability before and after digestion. As shown in FIG. 7d, the viability of SNaPs increased to 93.0±1.0% or 94.7±0.46% and 92.3±2.08% or 93.3±1.53% at day 1 and day 3 after digestion by 500 U mL⁻¹ or 1000 U mL⁻¹ of Hase, respectively. These results elucidated that the post-bioprinting enzymatic treatment of GelMA/HAMA constructs facilitated the proliferation and viability of SNaPs.

### Transcriptomic Analyses

Global transcriptome profiling was conducted through RNA sequencing (RNA-seq) on SNaPs after 4 weeks of differentiation (or 31 days after digestion) in the GM and Hase-1000 samples. No non-digested GH samples were compared since most cells were already dead at this time point of culture not allowing RNA-extraction. The heatmap presented differentially expressed genes (DEGs) between Hase-1000 and GM samples (FIG. 7e). The principal components analysis (PCA) implied that the GM group had a drastic transcriptional profile difference with the Hase-1000 samples (FIG. 7f). Overall, 715 genes were significantly upregulated while 256 genes were dramatically downregulated (Q-value <0.05, FIG. 7g). As shown in the volcano plot of FIG. 7h, the overexpressions of hexokinase 2 (HK-2) and phosphoinositide-dependent protein kinase 1 (PDK1) were found in the Hase-1000 group compared to the GM group, suggesting inhibited apoptosis and accelerated cell proliferation after Hase digestion. Lee, H. J. et al., Nat. Commun. 10, 2625 (2019). Moreover, neuronal-related genes were upregulated in the Hase-1000 group, such as stanniocalcin 2 (STC2), which is an injury-responsive gene required for axon regeneration, Jeon, Y. et al., Mol. Neurobiol. 58, 750-760 (2021); neuritin 1 (NRN1), which encodes extracellular glycosylphosphatidylinositol (GPI)-anchored protein to stimulate axonal plasticity, dendritic arborization, and synapse maturation, Zhou, S. & Zhou, J. Curr. Med Chem. 21, 1212-1219 (2014); and enolase 2 (ENO2), Stogsdill, J. A. et al., Nature 551, 192-197 (2017), which exhibits neurotrophic and neuroprotective properties in the central nervous system (CNS), Carletti, B. et al., Curr. Neuropharmacol. 9, 313-317 (2011).

To elucidate the biological processes within the different bioink formulations and treatment conditions, we performed both Gene Ontology (GO) and Kyoto Encyclopedia of Genes and Genomes (KEGG) pathway-enrichment analyses with the RNA-seq results of GM and Hase-1000 samples. The differences of transcriptional profiles in molecular functions and biological processes are displayed in FIG. 7i. Positive cell cycle regulation indicated an enhanced proliferation of SNaPs in the Hase-1000 group compared to the GM group, which was consistent with the SNaP proliferation results. Cellular processes related pathways including focal adhesion, gap junction, regulation of actin cytoskeleton, and adherens junctions were better-represented in the Hase-1000 samples compared to the GM samples. Modulation by these pathways would enhance cell adhesion, cell-cell interactions, and cell proliferation. Therefore, our transcriptome analyses revealed that the Hase-digestion process provided extra matrix spaces and thus improved the microenvironment for SNaP growth, maturation, and function.

We further focused on several mechanotransduction pathways to better understand the major differences for SNaP behaviors within GM and Hase-1000 groups. Among the enriched pathways, the hippo has been demonstrated as an essential signaling pathway in the survival and differentiation of neural stem cells (FIG. 7i), Mo, J. S. et al., EMBO Rep. 15, 642-656 (2014), activated by mechanical cues generated from altered cell shape, cell polarity, cell-cell junctions, or extracellular matrix (ECM) stiffness. During tissue regeneration or organ development, cells constantly respond to mechanical stress from neighboring cells and ECM, or shear force when they migrate. Ingber, D. E. FASEB J. 20, 811-827 (2006). These factors are transmitted via membrane receptors, actin cytoskeleton, and the nuclear membrane to influence gene expressions within the nuclei, resulting in changes not only in cell morphology and survival but also in cell fate-specification. Orr, A. W. et al., Developmental cell 10, 11-20 (2006). More importantly, the highly enriched terms related to the nervous system (FIG. 7i) illuminated that intended cell differentiation clearly happened more efficiently in the Hase-1000 samples. Specifically, a heatmap was produced to compare DEGs involved in the glutamatergic synapse pathway, revealing that genes GNB5, GNG7, ADCY8, GNG4, SLC17A7, MAPK3, GRIN3B, CACNA1A, ADCY5, GRIN1, DLG4, PRKCB, GRIK5, GLS2, PRKCG, ADCY7, GNG13, ADCY3, KCNJ3, and GRIN2D were upregulated in the Hase-1000 group. Gene set enrichment analysis (GSEA) results also elucidated that compared to the GM group, the Hase-1000 samples expressed enriched gene sets involved in synaptic signaling, neurotransmitter transport, and long-term synaptic potential pathways.

### Immunological Staining

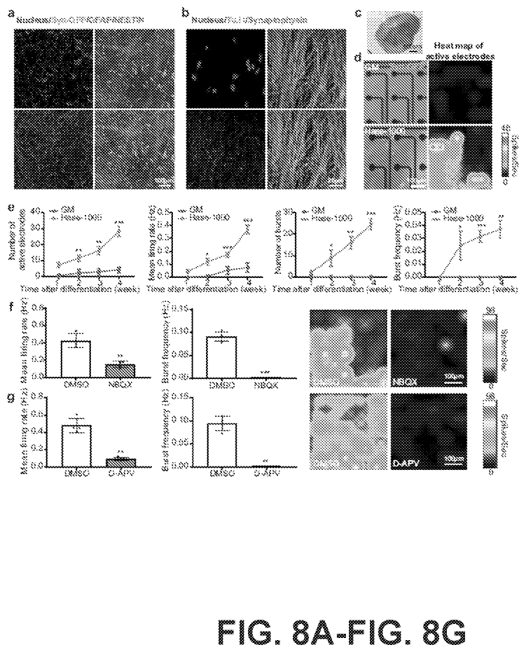

The ability to differentiate into multiple cell types of the neural and glial lineages is a hallmark of NPCs. Goldman, S. Nat. Biotechnol. 23, 862-871 (2005). To assess the differentiation capacities of SNaPs, we stained SNaPs in the bioprinted constructs for glial fibrillary acidic protein (GFAP), which is expressed by both immature and mature astrocytes as well as NPCs, Freeman, M. R. Science 330, 774-778 (2010), and nestin, as an immature neural marker, Romero-Ramos, M. et al., J. Neurosci. Res. 69, 894-907 (2002), during the process of differentiation. Immunostaining of 4-week-differentiated SNaPs (or 31 days after digestion) within the constructs presented the expression of neuronal marker (Syn-green fluorescent protein, GFP) and GFAP, but NESTIN+ cells were not detected (FIG. 8a), indicating the multipotency of the bioprinted SNaPs achieved through digestion of the GelMA/HAMA hydrogel. To distinguish between astrocytic differentiation and remaining in a precursor state, the S100 calcium binding protein B (S100B) staining was performed on the Hase-1000 sample to confirm the astrocytic differentiation of the bioprinted SNaPs within the Hase-digested GelMA/HAMA constructs. Steiner, J. et al., BMC Neurosci. 8, 2 (2007). Through staining of neuron-specific class III β-tubulin (Tuj1) and synaptophysin, we observed the characteristic punctate expression of the synaptic marker synaptophysin (FIG. 8b), which revealed that the bioprinted SNaPs were capable of differentiating into neurons and displaying functional synaptic proteins. Our findings suggested that these 3D-bioprinted SNaP-encapsulating constructs followed by post-bioprinting enzymatic digestion could enable the cells to differentiate into neurons and astrocytes, which are representative cell types of neuronal tissues.

Another promising finding was that the different matrix rigidities had a significant influence on SNaP cell differentiation. Immunostaining of 3D-bioprinted SNaPs showed that Hase-1000 inducted formation of both neurons (Syn-GFP) and astrocytes (GFAP), whereas only neurons were observed in the Hase-500 samples. The mechanical properties of the bioprinted constructs treated by Hase-500 and Hase-1000 were 3.98 kPa and 1.29 kPa, respectively, representing a notable difference in matrix rigidity. Different GelMA/HAMA formulations also exhibited apparent variations in subtype-specification after differentiation. Both 5.0% and 2.5% GelMA bioinks homogeneously mixed with 1.5% HAMA after 1000 U mL⁻¹ Hase digestion showed both astrocyte- and neuron-formation. By contrast, SNaPs bioprinted by the GelMA/HAMA (2.5%/3.0%) bioink possessing a relatively high stiffness after Hase digestion, exhibited the limited cell spreading and neuronal differentiation. These results tied well with previous studies wherein the mechanotransduction of neuronal cells, defined as the conversion of force a cell generates through cell-substrate bonds to a chemical signal, has great significance on neural tissue engineering. Uto, K. et al., Prog. Polym. Sci. 65, 53-82 (2017). Our findings confirmed that matrix rigidity is an essential biophysical cue affecting neuronal induction and subtype-specification, and suggested that 3D-bioprinted brain-mimics with native tissue-matching mechanical properties could thus serve as a promising platform for nervous system modeling.

### Electrophysiology Assessments

To access the electrophysiological functions of the SNaP-derived neurons in our bioprinted 3D mini-brains, multi-electrode array (MEA) was used to detect action potentials and validate the formation of functional synaptic networks. Bakkum, D. J. et al., Nat. Commun. 4, 2181 (2013). Neurons in Hase-1000 and GM samples were separately placed on the electrode arrays of MEA plates and the electrophysiological signals were recorded every week for up to 4 weeks after differentiation (FIG. 8c-e). Also note again, the readily maintained overall shape of the bioprinted mini-brain after digestion and culture, shown in FIG. 8c. The average number of active electrodes increased over time and was higher in neurons of the Hase-1000 group than in those of the GM group (FIG. 8d-e), indicating the increased number of active neurons in the Hase-1000 samples. The mean firing rate followed a similar time course-dependent increase and was significantly elevated in neurons in the Hase-1000 samples. Burst analyses of the MEA recordings identified synchronous firing events as bursts with a burst frequency detectable from as early as week 2. Notably, the number of bursts and burst frequency increased with time in the Hase-1000 samples, but neurons within the GM group showed negligible burst events, confirming the inability of neurons to form synaptic networks in the GM hydrogel.

We finally confirmed the synaptic contributions to burst activities in the Hase-1000 samples at week 4 after culture onto the MEA plates through pharmacological interventions. The mean firing rate and burst frequency were significantly reduced by 2,3-dihydroxy-6-nitro-7-sulfamoyl-benzo(F)quinoxaline (NBQX, FIG. 8f), an α-amino-3-hydroxy-5-methyl-4-isoxazole-propionate (AMPA) receptor-antagonist, Pitt, D., et al. Nat. Med. 6, 67-70 (2000), suggesting that the neuronal network activity by the cells in the Hase-1000 group was driven in part by excitatory synaptic transmission mediated by ionotropic glutamate receptors. Similarly, the addition of D-2-amino-5-phosphonovaleric acid (D-APV), an N-methyl-D-aspartate (NMDA) receptor-antagonist, Murphy, G. G. & Glanzman, D. L. J. Neurosci. 19, 10595-10602 (1999), significantly reduced the firing and burst frequency (FIG. 8g). Our electrophysiological results demonstrated that the Hase-1000 treatment promoted the synaptic activity and network formation of SNaP-induced neurons, which was consistent with our transcriptomic profiling data as well as related analyses. Overall, the difference in gene expression and subsequent functional changes of cells differentiated in the post-bioprinting enzymatically digested mini-brains compared to the control were likely combined results of the dimensionality and tunable mechanical cues of the ECM-like matrix, as well as favored cellular crosstalk of SNaPs.

Collectively, we observed from our series of studies that the presence of HAMA, although strengthening printability, might pose unwanted negative impacts on cellular growth and functions especially when soft tissues are involved, since addition of HAMA would inevitably result in densely crosslinked hydrogel networks. We hence proposed that rational utilization of Hase could cleave the HAMA molecules in the bioprinted HAMA/GelMA constructs and reduce the crosslinking densities of the entire hydrogel networks. As a result, mechanical stiffness of the bioprinted constructs could be precisely tuned, where the cleaved HAMA also provided cells with more space to grow and favored their functions, such as stem cell differentiations otherwise not readily attainable in similar hydrogel systems.

In conclusion, we report the utilization of a GelMA/HAMA bioink as a generalized methodology, which may transform the field of DLP bioprinting. This approach offers unprecedented bioprinting possibilities where designed constructs could be printed at high structural sophistication and fidelity, and post-printing enzymatically digested for the HAMA component to meet mechanical and biological requirements of target tissues. These two requirements are usually contradictory posing a major challenge in conventional DLP-based bioprinting, which is successfully resolved using our extremely simple yet powerful molecular cleavage approach. We have established an extensive library of mechanical parameter maps based on both experimental results and theoretical modeling, resulting in broadly tunable and precisely controllable mechanical properties by adjusting bioink formulations and digestion conditions.

Our demonstration of this enabling DLP-bioprinting strategy presented the excellent printability of tissue-mimicking structures featuring sophisticated shapes and internal architectures, which were well-retained after enzymatic digestion. Importantly, our results provided evidence of cell morphological and functional enhancements of encapsulated cells, suggesting the suitability of this method for the bioprinting of living constructs of soft-tissue origins, such as the brain, liver, and muscles. Of special interest, as illuminated by immunocytochemistry and RNA-seq data, SNaPs in our DLP-bioprinted volumetric mini-brain tissues differentiated into neurons and astrocytes; these neurons exhibited strong electrophysiological and other brain-like properties, which are extremely hard to achieve before. The broad implication of the present research lies in expanding the palette of bioinks usable for high-fidelity DLP bioprinting, enabling the biofabrication of mechanically tunable constructs to meet the biological function requirements of target tissue-mimics, in particular those that are soft and ultrasoft. Our technology does not come without limitations. For example, the careful balance between enzymatic digestion conditions and cellular viability would need to be considered when applying it to practice, among others. With further optimizations, this unique molecular cleavage strategy relying on post-bioprinting enzymatic treatment is anticipated to find widespread applications in tissue and tissue model engineering.

### Methods

### GelMA Synthesis and Characterizations

GelMA was synthesized according to previously reported methods. Maharjan, S. et al., Matter 4, 217-240 (2021). In brief, type A gelatin from cold-water fish skin (10 g, M_{w}=60 kDa, Sigma-Aldrich, USA) was dissolved in phosphate-buffered saline (100 mL, PBS, ThermoFisher, USA) at the concentration of 10%. Methacrylate anhydride (12 mL, MA, Sigma-Aldrich) was then added dropwise and was reacted with gelatin solution at 50° C. for 2 h. After diluting the solution 1:1 with warm PBS (40° C.) to stop the reaction, dialysis was conducted for 7 days against deionized (DI) water using dialysis membrane (M_{w} cut off (MWCO): 12-14 kDa, Spectrum Chemical, USA) at 40° C. to remove low-molecular weight impurities. Subsequently, the solution was lyophilized and stored at −20° C. in dark until use. The lyophilized GelMA was further characterized by ¹H NMR spectroscopy (Bruker Avance II 300-MHz NMR, USA). NMR data were processed with the MestreNova (6.2.0) software. The primary amine contents of gelatin and synthesized GelMA were evaluated by TNBSA assay. Zhu, M. et al., Sci. Rep. 9, 1-13 (2019).

### HAMA Synthesis and Characterizations

The synthesis of HAMA followed the procedure as previously described. Gong, J. et al., Nat. Commun. 11, 1267 (2020). Briefly, 4.0 g of HA with different M_{w} ranging from 10 to 1500 kDa (10, 60, 100, 200, 500, or 1,500 kDa, HA works, USA), was fully dissolved in 200 mL of DI water at 4° C. 133.3 mL of dimethylformamide (DMF, Sigma-Aldrich) and 7.88 mL of MA were added into HA solution under vigorous stirring. The pH of the solution was regulated to pH 8-9 with 1-M sodium hydroxide (Sigma-Aldrich) solution. The reaction was kept at 4° C. under continuous stirring for another 18 h. Subsequently, 0.5-M NaCl (Sigma-Aldrich) was dissolved in the mixture, and the mixture was precipitated in a doubled volume of ethanol (Sigma-Aldrich). HAMA was then collected as white pellets after precipitation. The precipitate was washed with ethanol for 3 times before being dissolved in DI water and the solution was dialyzed against DI water for 5 days. The purified product was obtained by lyophilization and characterized by ¹H NMR spectroscopy.

### FITC-Conjugated HAMA (HAMA-FITC) Synthesis

To conjugate FITC to HAMA, we first dissolved 1.0 g of HAMA (100 kDa) in 2-(N-morpholino)ethanesulfonic acid (MES) buffer (pH=5.6, Sigma-Aldrich). Then, the 0.1-M N-(3-dimethylamino propyl)-N-ethylcarbodiimide (EDC, Sigma-Aldrich) and 0.2-M N-hydroxysuccinimide (NHS, Sigma-Aldrich) mixture was added to activate the carboxylate groups on HAMA. 10-μg mL⁻¹ FITC-poly(ethylene glycol)-amine (FITC-PEG-NH2, M_{w}=2 kDa, Nanocs, USA) was added and reacted overnight at 4° C. in dark. The reacted solution was dialyzed against DI water for 5 days and then lyophilized for use.

### DLP-Based 3D Bioprinter

A DLP-based 3D bioprinter was built in-house using a projection device, Miri, A. K. et al. Adv. Mater. 30, 1800242 (2018), the PRO4500 Optical Engine, with a display resolution of 912×1140 pixels (Wintech Digital Systems Technology, USA). The optical pattern generated by the projector was reflected by an aluminum front-coated mirror (Edmund Optics, USA) placed at 450 and focused to obtain the field of view of 65.6×41 mm² and an x-y resolution of 50 μm. A build platform hosting a glass slide (Carolina, USA) allowed the printed construct to attach during and after printing, which was controlled by a stepper motor linear drive (TOAUTO, USA) along the z-axis. Custom software was developed in MATLAB (v2020, MathWorks, USA) to control the DLP bioprinting processes. A Teflon film (Random Technologies, USA) was used as the bottom of the vat to enable a clear transmission of light and to provide an oxygen-permeable window. Multiple printing models used in this study, including the cube (FIG. 4a), the pyramid (FIG. 4c), the cuboid with a spiral channel (FIG. 4e), the hepatic lobule (FIG. 5e), and the lung (FIG. 5e), were designed in SolidWorks (Dassault Systemes, France), whereas those of the gyroid (FIG. 4d), the brain (FIG. 5e), and the heart (FIG. 5e), were acquired from Thingiverse (MakerBot Industries, USA) and reused with the permission of their creators. The model of the torus knot in FIG. 4f was obtained from a previous study. Grigoryan, B. et al. Science 364, 458-464 (2019). Each designed digital file was sliced into a series of two-dimensional (2D) images at a thickness of 100 μm using the open-source DLP slicer.

### 3D Printing Procedure

The desired ink was transferred into the vat before printing, followed by lowering the build platform to the initial printing position. The full control over the image projection and the movement of the build plate was realized by our customized software. Li, W. et al., Adv. Mater. 33, 2102153 (2021). After printing was completed, the 3D-printed construct was removed from the build platform and washed in PBS multiple times to remove the uncrosslinked ink. For visualizing the internal hollow structures within the printed 3D constructs, we perfused the painting pigment (Easyou, China) solution into the open channels. To demonstrate the possibility of complex printing structures using the enzymatically digestible inks, the ink formulated with GelMA/HAMA (5.0%/3.0%, M_{w}=100 kDa), 1-mM/10-mM tris(2,2-bipyridyl)dichlororuthenium(II) hexahydrate (Ru)/sodium persulfate (SPS) (Advanced BioMatrix, USA), and 2.0% photoabsorber (Ponceau, Sigma-Aldrich) was selected as an example. The pyramid (4×4×4 mm³), the gyroid (8×8×8 mm³), the cuboid containing a spiral channel (10×5×4 mm³), and the torus knot (18-mm length and 5-mm height) were printed with 15 s of exposure time for each layer (100 m), in 10 min, 20 min, 10 min, and 12.5 min, respectively.

### Measurements of Working Curves of GelMA/HAMA Inks

The working curves of GelMA/HAMA inks were measured following the method as previously reported. Li, W. et al. Adv. Mater. 33, 2102153 (2021). A square pattern (4×4 mm²) was printed on the cover glass using the inks of GelMA/HAMA (5.0%/3.0%) and 1-mM/10-mM Ru/SPS without or with the photoabsorber (0.0, 1.0, 2.0, and 3.0% Ponceau), under the exposure times between 5 s and 30 s. Optical images of the thicknesses of the printed constructs were captured using a microscope (Nikon, Japan) and measured with ImageJ (v2.0.0-rc-69/1.52p, National Institutes of Health, USA).

### Printability and Resolution Evaluations of GelMA/HAMA Inks

To evaluate bioink printability, HAMA stock solutions with different M_{w} were prepared by dissolving into PBS to be 1.0% HAMA (M_{w}=1500 kDa) or 5.0% HAMA (M_{w}=60, 100, 200, or 500 kDa). GelMA was dissolved in PBS as a stock solution of 20%. HAMA and GelMA solutions were mixed into the final formulations of 0.25, 0.50, 0.75, 1.00, 1.25, 1.50, 1.75, 2.00, 2.25, 2.50, 2.75, or 3.00% HAMA, and 2.5, 5.0, or 7.5% GelMA. Additionally, 1-mM/10-mM Ru/SPS and 2.0% photoabsorber were selected based on desired layer thicknesses. To fabricate the 3D cubic construct used to assess the printability, a cube of 4×4×4 mm³ was printed with a 100-μm thickness and a 30-s printing time for each layer.

The printing resolutions of GelMA/HAMA inks were tested using a standardized evaluation model and method as previously reported. A radial pattern formed by ten lines with a central circle design was printed on the cover glass. The diameter of the central circle pattern was derived from the width and the number of lines. Printing resolution (p), defined as the distance between two recognizable independent points, can be calculated by Eq. (4):

$$p = D \times tan \frac{π}{n} - \frac{h}{cos \frac{π}{n}}$$

where D is the diameter of the printed central circle measured from the optical image of printed structure, n is the number of radial lines, and h is the width of the designed lines. The inks of GelMA/HAMA (5.0%/3.0%) and 1-mM/10-mM Ru/SPS without or with 2.0% photoabsorber were printed under the exposure times between 2.5 s and 30 s. Optical images of the printed constructs were captured using a microscope and measured with ImageJ.

### Measurements of Mechanical Properties

To measure the mechanical properties of hydrogels made from different inks, the inks were cast in polydimethylsiloxane (PDMS, Dow, USA) molds (4 mm in width, 3 mm in length, and 3 mm in height) and subsequently photocrosslinked. The compressive moduli of GelMA/HAMA constructs before and after Hase (Sigma-Aldrich) digestion were conducted as described before. Gong, J. et al., Nat. Commun. 11, 1267 (2020). Compression tests were performed on a 6800 SERIES mechanical tester (Instron, USA) with a ramp of 2.0 N min⁻¹ up to a maximum of 100.0 N. Bluehill Universal (v4.13, Instron) and origin (v9.8, OriginLab, USA) were used to analyze the mechanical tests data. The compressive modulus was calculated according to the slope of the stress-strain curve within the linear region between 0 and 20% strain.

### Enzymatic Digestion of GelMA/HAMA Constructs

HAMA of 100 kDa in M, was selected to investigate the effects of Hase digestion on the mechanical properties of printed constructs. The constructs with the same size (4×4×2 mm³) fabricated by 1.5% or 3.0% HAMA and 2.5%, 5.0%, or 7.5% GelMA were immersed in Hase solutions (0, 75, 150, 300, 500, or 1000 U mL⁻¹) for different treatment times (0, 1, 2, 4, 8, 12, or 24 h) under 37° C. with shaking. All the digested constructs were measured for their mechanical properties as described above. For the visualization of digestions, 1.5% HAMA-FITC was used to combine with 2.5%, 5.0%, or 7.5% GelMA. The samples were printed onto cover glasses with the same size (4×4×1 mm³) and transfer into the wells of 24-well plates for post-printing enzymatic digestion. The Hase solutions were prepared with different concentrations of 0, 75, 150, 300, 500, and 1000 U mL⁻¹, or different digestion durations of 0, 1, 2, 4, 8, 12, and 24 h under 37° C. Fluorescence micrographs were captured at the corresponding time points, and the fluorescence intensities were analyzed with ImageJ.

The diffusion coefficient of Hase was measured following previous methods. Hettiaratchi, M. H. et al., APL Bioeng. 2, 026110 (2018). Briefly, hydrogel samples were prepared by photocuring the GelMA/HAMA (5.0%/3.0%) bioink in rectangular cuvettes (2×10×45 mm³). The obtained samples sealed in cuvettes were first treated with PBS and Hase (1000 U mL⁻¹) for 24 h at 37° C. To measure the diffusion coefficients, 2 mg mL⁻¹ of FITC-dextran (M_{w}=60 kDa) was added to the top of the cured hydrogel in each cuvette. Then, the fluorescence images of samples were taken after contacting with FITC-dextran for 1 min using a fluorescence microscope (Zeiss, Germany). The fluorescence intensities were analyzed by ImageJ. To normalize the fluorescence intensities, the fluorescence intensity at the opening side of cuvette was set to 1, and the value at the end of cuvette was set to 0. The diffusion through the hydrogel could be described using the following Eq. (5),

$$F ∝ erfc ( \frac{x}{2 \sqrt{{D}_{eff} t}} )$$

where erfc is the complementary error function, F is the normalized fluorescence intensity, x is the distance from the opening side (cm), t is the time since the FITC-dextran and hydrogel contact (s), and D_{eff} is the effective diffusion coefficient (cm² s⁻¹).

### Mathematical Modeling

The mechanical properties of fabricated constructs were primarily affected by the GelMA concentration (%), the HAMA concentration (%), the Hase concentration (U mL⁻¹), and the digestion duration (h). A mathematical model was used to describe and predict the moduli of the constructs based on these parameters. The effects of these parameters on the moduli were represented by the mechanical data from the experiments, which were then fitted with a quadratic function to create response surfaces. The model can then be used to approximate and explore the functional surfaces between response variables and a set of design factors. Ferrenberg, S. et al., Proc. Natl. Acad. Sci. USA 112, 12116-12121 (2015). In this study, we applied regression modeling and optimization methods to the historical data to determine the best model to fit the system.

The compressive modulus (kPa) was correlated with the experimental parameters A: GelMA concentration (%), B: digestion time (h), C: Hase concentration (U mL⁻¹), and D: HAMA concentration (%). A second-degree polynomial model Eq. (6) was fitted to the experimental data.

$$Y = {a}_{0} + {\sum }_{i} {b}_{i} {x}_{i} + {\sum }_{i , j} {c}_{ij} {x}_{i} {x}_{j}$$

where Y was the predicted response, xᵢ, and xⱼ, were experimental parameters, and a₀, bᵢ, and cᵢⱼ were constant, linear, and quadratic coefficients, respectively. When i≠j, these were interaction coefficients. The signal-to-noise ratio of the model was quantified by adequate precision, which compared the range of predicted values at the experimental points to average prediction error. Excellent accuracy of >4.0 indicated that the model was suitable to navigate the design space for prediction.

### Collagenase Digestion of GelMA in GelMA/HAMA Constructs

GelMA/HAMA (2.5%/1.5%, M_{w} of HAMA=100 kDa) and GelMA/HAMA (2.5%/2.0%%, M_{w} of HAMA=100 kDa) were selected to demonstrate the feasibility of digesting GelMA molecules with collagenase and obtaining softer, HAMA-rich hydrogels. The fabricated constructs were immersed in collagenase type II solution (Worthington Biochemical, USA) of 0, 1, and 2 U mL⁻¹ for 24 h under 37° C. with shaking. All the digested constructs were measured for their mechanical properties as described above.

### Evaluation of Cell Leaching

The constructs (cubes of 4×4×2 mm³) were bioprinted with a 400-μm thickness and a 30-s printing time for each layer using the bioink of GelMA/HAMA (5.0%/3.0%) containing NIH/3T3 fibroblasts (6×10⁶ cells mL⁻¹, CRL-1658, American Type Culture Collection (ATCC), USA). After bioprinting, the samples were transferred into the wells of a 24-well plate for enzymatic digestion in 1000 U mL⁻¹ of Hase for 24 h. The images of leached cells in the wells were captured using a microscope and the leached cell numbers were quantified with ImageJ. Moreover, cellular metabolic activities were assessed by the 3-(4,5-dimethylthiazol-2-yl)-5-(3-carboxymethoxyphenyl)-2-(4-sulfophenyl)-2H-tetrazolium (MITS) assay using the CellTiter 96@AQueous Assay (Promega, USA) according to the manufacturer's instructions. Briefly, the samples were incubated with the MTS assay reagent for 4 h in the dark. Subsequently, the absorbance values were measured at 490 nm with a microplate reader (Molecular Devices, USA).

### Fabrication and Characterizations of Tissue-Mimics

The tissue mimics were printed with selected inks at 15-s exposure time for each layer (100 m of layer thickness), where 2.5%, 5.0%, 5.0%, or 7.5% GelMA containing 1.5% or 3.0% HAMA, 1-mM/10-mM Ru/SPS, and 2.0% photoabsorber were applied to print the brain-like (8-mm length and 5-mm height), liver-like (8×6×5 mm³), lung-like (6×6×6 mm³), or heart-like (8-mm height) structure, respectively. To achieve tissue-specific mechanical properties, the fabricated constructs were treated with Hase of 1000 U mL⁻¹ for 24 h for the brain-, liver-, and heart-mimics, but with Hase of 500 U mL⁻¹ for 24 h for the lung-mimic. SEM (Zeiss, Germany) was used to visualize the microstructures of the printed constructs before and after Hase digestion after lyophilization. Zeiss SmartSEM software (v05.07 SP4) was used to acquire SEM images. In addition, the pore size and surface area were further measured by a Brunauer-Emmett-Teller (BET) surface area analyzer (NOVA 2200E, Quantachrome, USA). The data was obtained from nitrogen-adsorption at 77 K. Before adsorption, all samples were prepared by degassing at 80° C. for 48 h. BET surface area and Barrett-Joyner-Halenda (BJH) cumulative pore volume/distribution were measured and analyzed by the NOVA software.

### Muscle Tissue-Bioprinting and Characterizations

C2C12 mouse skeletal myoblasts (CRL-1772, ATCC) was cultured in Dulbecco's modified Eagle medium (DMEM, ThermoFisher) supplemented with 10 v/v % fetal bovine serum (FBS, Gibco, USA) and 1 v/v % antibiotic-antimycotic (Gibco). C2C12 myoblasts (passages 3-6, 6×10⁶ cells mL⁻¹) were mixed with GelMA/HAMA (7.5%/1.5%) and 1-mM/10-mM Ru/SPS as the bioink. To make the imaging evaluations of bioprinted tissues easier, cubes of 4×4×2 mm³ were bioprinted with a 400-μm thickness and a 30-s printing time for each layer. After bioprinting, the constructs were transferred into the wells of a well plate for enzymatic digestion in 1000 U mL⁻¹ of Hase solution for 24 h, and then replaced by complete culture medium.

To induce differentiation, constructs containing C2C12 myoblasts were starved of serum and cultured in DMEM containing 2 v/v % horse serum (ThermoFisher) at 3 days after Hase digestion. The differentiation medium was changed every day to provide enough nutrition for cell growth. At 7 and 14 days after differentiation, the samples were fixed with 4% paraformaldehyde (Sigma-Aldrich) for 15 min, permeabilized with 0.03 v/v % Triton X-100 (Sigma-Aldrich) for 10 min, and then blocked by 5% bovine serum albumin (BSA, Sigma-Aldrich) for 1 h at room temperature. The samples were incubated with the primary antibody of MHC (Abcam, USA, dilution 1:100, cat no. ab91506) overnight at 4° C., and then incubated with the secondary antibody (Alexa Fluor 488 goat anti-rabbit IgG, dilution 1:500, ThermoFisher, cat no. A32731) at 37° C. for 2 h. 4′, 6-diamidino-2-phenylindole (DAPI, Vector Laboratories, USA, dilution 1:2000, cat no. D1306) was finally used to counterstain the nuclei. The stained samples were visualized by a confocal microscope (LSM880, Zeiss). The acquired images from ZEN Black 2.1 (Zeiss) were analyzed with ImageJ. The fusion index was calculated as the ratio of the nucleus number for cells with two or more nuclei to the total number of nuclei.

### Liver Tissue-Bioprinting and Characterizations

We bioprinted the liver tissue-mimic out of bioink compositions of GelMA/HAMA (5.0%/1.5%) or 10% GelMA only (minimally printable concentration of GelMA for DLP in the absence of HAMA), and 1-mM/10-mM Ru/SPS, using 8×10⁶ cells mL⁻¹ of HepG2/C3A cells (CRL-10741, ATCC). The bioprinted constructs (cubes of 4×4×2 mm³) were transferred into the wells of a well plate, where DMEM containing FBS in the absence or presence of Hase solution at 1000 U mL⁻¹ was added and incubated at 37° C. for 24 h. At 1, 3, and 7 days after Hase digestion, the viabilities of the cells were measured with live/dead staining (Invitrogen, USA). In detail, the constructs were rinsed with PBS and incubated with 2 μM of calcein-AM and 4 μM of ethidium homodimer-1 for 30 min and then observed by fluorescence microscopy. The numbers of live cells and dead cells were quantified with ImageJ. Additionally, cellular metabolic activities were assessed using MTS assay with the CellTiter 96® AQueous Assay. Moreover, the supernatants from cell cultures at 5, 10, and 15 days after Hase digestion were collected and stored at −80° C. The quantifications of ALB and urea secretions were conducted using an enzyme-linked immunosorbent assay (ELISA) kit for ALB (Abcam) and urea assay kit (Sigma-Aldrich), respectively, according to the manufacturers' instructions.

The samples were collected at 7 and 14 days after Hase digestion for the evaluations of gene expressions. The samples were immersed in TRIzol (ThermoFisher) and homogenized using the Precellys lysing kits (Precellys, France) to isolate the total RNAs. Then, the first-strand cDNA was synthesized using the SuperScript® VILO™ cDNA Synthesis Kit (Invitrogen) according to the instructions of the manufacturer. The quantitative reverse transcription-polymerase chain reaction (qRT-PCR) was conducted with the PowerTrack SYBR Green Master Mix (ThermoFisher). The primers were obtained from Integrated DNA Technologies (USA). RT-PCR analysis was then run on the QuantStudio 5 Real-Time PCR instrument (ThermoFisher) and in QuantStudio Real-Time PCR Software (v1.5.1, Applied Biosystems, USA) with duplicate copies and the results were normalized against the housekeeping GAPDH gene. For immunostaining, the samples were fixed, permeabilized, and blocked by 4% paraformaldehyde, 0.03 v/v % Triton X-100, and 5% BSA, respectively. Subsequently, the samples were incubated with the primary antibodies against ALB (Abcam, dilution 1:500, cat no. ab207327), E-cadherin (Abcam, dilution 1:500, cat no. ab231303), CYP1A2 (Abcam, dilution 1:500, cat no. ab22717), or CYP3A4 (Abcam, dilution 1:250, cat no. MA3-032) overnight at 4° C., and then incubated with the corresponding secondary antibody (Alexa Fluor®-488 goat anti-rabbit IgG, cat no. A32731; Alexa Fluor®-594 donkey anti-mouse IgG, cat no. A32744; Alexa Fluor®-594 donkey anti-mouse IgG, cat no. A32744; Alexa Fluor®-488 goat anti-mouse IgG, cat no. A32723; dilution 1:500) at 37° C. for 2 h, which was followed by DAPI counterstaining of the nuclei. The samples were then visualized using a confocal microscope, and the integrated optical densities (IODs) of fluorescence were analyzed with ImageJ.

Casting hydrogel constructs encapsulating HepG2/C3A cells or C2C12 myoblasts

The 5% GelMA solutions containing HepG2/C3A cells (8×10⁶ cells mL⁻¹), or the 7.5% GelMA solutions containing C2C12 myoblasts (6×10⁶ cells mL⁻¹) were cast in rectangle PDMS molds (4×4×2 mm³) and subsequently crosslinked through exposure to ultraviolet (UV) irradiation (10 mW cm⁻², 360-480 nm, 40 s, OmniCure S2000, Excelitas, USA). At 1, 3, and 7 days after culture, the viabilities of the HepG2/C3A cells were measured with live/dead staining and then observed by fluorescence microscopy. The numbers of live cells and dead cells were quantified with ImageJ. Moreover, cellular metabolic activities were assessed by MTS assay with the CellTiter 96® AQueous Assay. The C2C12 myoblast samples were collected on day 3 after the casting and were stained with Alexa Fluor 594 Phalloidin (ThermoFisher, dilution 1:400, cat no. A12381) for F-actin observation. The images of C2C12 myoblasts under bright-field and fluorescence were then captured using a microscope.

Brain Tissue-Bioprinting with SNaPs and Characterizations

Human embryonic stem cells (hESCs) were cultured and maintained with mTeSR plus medium (Stem Cell Technologies, USA) on Geltrex (ThermoFisher)-coated tissue culture dishes. H01 hESCs were maintained in 5% CO₂ incubators at 37° C. and passaged every 4-5 days as small aggregates after accutase (Innovative Cell Technologies, USA)-treatment. After dissociation, 10-μM ROCK-inhibitor (Sigma-Aldrich) was added to the cell culture for 24 h to prevent cell death. For NGN2 viral transduction, TetO-Ngn2-Puromycin and Ubq-rtTA plasmid constructs were obtained from the Wernig Lab and packaged as high-titer lentiviruses (Alstem, USA). The cells were dissociated and resuspended in virus-containing mTeSR medium supplemented with ROCK-inhibitor at a multiplicity of infection (MOI) of 1 to 3. Transduced cells were maintained in mTeSR for up to ten passages, with a 70-90% transduction efficiency.

The protocol for inducing SNaPs was previously described. Wells M. F. et al., bioRxiv 116, 9527-9532 (2018). In brief, the cells were maintained in mTeSR medium and were fed with induction medium containing DMEM/F12 (ThermoFisher) supplemented with 20% glucose (1.5 v/v %, ThermoFisher), 1:100 glutamax (ThermoFisher), 1:100 N2 supplement (ThermoFisher), 2 μg mL⁻¹ of doxycycline (Sigma-Aldrich), 10 μM of SB431542 (Tocris, UK), 200 nM of LDN-193189 (Stemgent, USA), and 2 μM of XAV939 (Stemgent) on day 1. After 24-h induction, medium was changed to selection medium with DMEM/F12, 20% glucose (1.5 v/v %), 1:100 glutamax (ThermoFisher), 1:100 N2 supplement, 2-μg mL⁻¹ doxycycline, 5-μg mL⁻¹ puromycin (ThermoFisher), 100-nM LDN-193189, 5-μM SB431542, and 1-μM XAV939 for 24 h. On day 3, SNaPs were dissociated and replated at a density of 1.2×10⁵ cells cm⁻² and maintained in SNaP maintenance medium using DMEM/F12 supplemented with 1:100 glutamax, 1:100 N2 supplement, 1:100 MEM-NEAA (ThermoFisher), 1:50 B27 minus vitamin A (ThermoFisher), 10-ng mL⁻¹ recombinant human basic fibroblast growth factor (bFGF, ThermoFisher), and 10-ng mL⁻¹ recombinant human epidermal growth factor (EGF, R&D Systems, USA). On the same day, 5-μg mL⁻¹ puromycin and 10-μM Y-27632 (Tocris) were added to the medium. About 12-24 h after replating, SNaPs were fed with a maintenance medium and passaged every 5-7 days for future experiments. For spontaneous differentiation, SNaPs were differentiated in base differentiation medium containing DMEM/F12 supplemented with 1:50 glutamax, 1:100 MEM-NEAA, 1:50 B27, 1:100 N2 supplement, and 10% FBS. 2-μg mL⁻¹ doxycycline was added to the base differentiation medium for 2-3 days to direct neuronal differentiation. The medium was then exchanged to remove doxycycline, and cells were fed 2-3 times a week using base differentiation medium supplemented with 10-ng mL⁻¹ brain-derived neurotrophic factor (BDNF, ThermoFisher), 10-ng mL⁻¹ ciliary neurotrophic factor (CDNF, ThermoFisher), and 10-ng mL⁻¹ glial cell line-derived neurotrophic factor (CNTF, ThermoFisher).

For the bioprinting of SNaPs, the cells (1×10⁷ cells mL⁻¹) were mixed with GelMA/HAMA (2.5%/1.5%) and 1-mM/10-mM Ru/SPS as the bioink. The bioprinting was conducted with a 30-s exposure time for each layer (400 μm of layer thickness) to achieve the final construct in 150 s. The bioprinted constructs (cubes of 4×4×2 mm³) were rinsed with PBS three times before adding Hase (500 or 1000 U mL⁻¹) for enzymatic digestion in the maintenance medium for culture. By contrast, the bioink of 10% GelMA (minimally printable concentration of GelMA for DLP in the absence of HAMA), which has been widely used in 3D bioprinting of brain-mimics (though again, at this concentration the cell functions would be very limited as also shown in literature, Tang, M. et al. Cell Res. 30, 833-853 (2020)) containing 1-mM/10-mM Ru/SPS was adopted as the control group. The Hase was removed at 24 h after digestion, and the bioprinted SNaPs were cultured in the maintenance medium for 3 extra days with medium changed every day.

The bioprinted cells were stained for Ki67 as a proliferation marker. Urruticoechea, A. el al., J. Clin. Oncol. 23, 7212-7220 (2005). The samples were fixed in 4% paraformaldehyde for 15 min, permeabilized with 0.03 v/v % of Triton X-100, and then blocked with 5% BSA for 1 h at room temperature. After being incubated with Ki67 antibody conjugated to Alexa Fluor® 594 (Abcam, dilution 1:100, cat no. ab216709) overnight at 4° C., or nestin antibody (Stem cell technologies, dilution 1:1000, cat no. 60091) overnight at 4° C. and secondary antibody (Alexa Fluor®-594 goat anti-mouse IgG, dilution 1:500, cat no. A32744) at 37° C. for 2 h, the stained samples were observed with the confocal microscope. The number of Ki67+ cells was quantified by ImageJ. Moreover, live/dead staining was carried out on days 1, 2, and 3 after digestion. The SNaP differentiation was induced at day 3 after Hase digestion by adding the differentiation medium, which was changed every 3 days during culture. To evaluate the differentiation of bioprinted SNaPs, the samples were fixed, permeabilized, and blocked with 4% paraformaldehyde, 0.03 v/v % Triton X-100, and 5% BSA, respectively. The samples were incubated with the primary antibodies of GFAP (Abcam, dilution 1:100, cat no. AB5804), TUJ1 (Abcam, dilution 1:1000, cat no. NL1195V), synaptophysin (Sigma-Aldrich, dilution 1:100, cat no. ZRB1365), or S100B (Sigma-Aldrich, dilution 1:1000, cat no. S2532) overnight at 4° C., and then incubated with the corresponding secondary antibody (Alexa Fluor©-647 donkey anti-rabbit, cat no. A-31573; Alexa Fluor©-488 goat anti-mouse IgG, cat no. A32744; Alexa Fluor©-488 goat anti-rabbit IgG, cat no. A32731; or Alexa Fluor®-488 goat anti-mouse IgG, cat no. A32723; dilution 1:500) at 37° C. for 2 h, which was followed by DAPI staining for the nuclei. The samples were then visualized using confocal microscopy.

MEA recording of 3D-bioprinted mini-brains was performed from week 1 to week 4 after differentiation (day 10 to day 31 after digestion). The samples containing SNaPs were plated on Geltrex-coated 12-well MEA plates (Axion Biosystems, USA) in the differentiation medium. The samples were fed 2-3 times a week with partial medium change. Neuronal activities were measured every week using the Maestro 12-well MEA plate system (Axion Biosystems) for 5 min. After 4 weeks of culture in MEA plate, synaptic contents were tested using pharmacological inhibitors of neurotransmitter receptors. Specifically, neuronal activity was measured for 5 min by adding DMSO control before adding 10 μM of NBQX (Abcam) or 10 μM of D-APV (Abcam). The samples were incubated for 5-10 min, and neuronal activities were assessed again for 5 min. All the MEA data were analyzed using the Axion Integrated Studio 2.4.2 and the Neural Metric Tool (Axion Biosystems).

### RNA-Seq and Data Analyses

Using TRIzol and Precellys lysing kits, RNA was extracted from 3D-bioprinted mini-brain samples, which were cultured for 28 days after differentiation. RNA-seq was processed and analyzed by Beijing Genomics Institute (BGI, China). Library quality and quantitation were conducted using a Qubit fluorometer (ThermoFisher) and an Agilent 2100 Bioanalyzer (Agilent Technologies, USA). After quality control, the constructed libraries were amplified with phi29 to make DNA nanoball (DNB), which had >300 copies of one molecular. The DNBs were loaded into the patterned nanoarray and single-end 50 (pair-end 100/150) bases reads were generated in the way of combinatorial Probe Anchor Synthesis (cPAS). We applied Bowtie2 for mapping the clean reads to the reference gene sequence, and then used RSEM to calculate the gene expression level of each sample. Significant DEGs were determined by false discovery rate (FDR)<0.05. According to the results of differential gene detection, the R package heatmap was used to perform hierarchical clustering analysis on the union set differential genes. PCA was performed for comparison between the samples (GM and Hase-1000). For GO and KEGG pathway enrichment analyses, all DEGs were mapped to terms in the KEGG and GO databases and queried for significantly enriched terms. Pathway with Q-value (corrected P-value)<0.05 was defined as the pathway that is significantly enriched in differentially expressed genes. GSEA was performed with the database of GSEA MSigDB C5 (GO) biological processes.

### Statistical Analyses

The data are presented as means±standard deviations (SDs). All statistical analyses were performed with one-way ANOVA followed by a Tukey's Honest Significant Difference test or a two-tailed student's t-test. p<0.05 was considered statistically significant. GraphPad Prism software (v8.2.0) was used for all statistical analyses, and Microsoft excel (v16.54) was used for data handling.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A bioink formulation for three-dimensional bioprinting comprising a mixture of a biocompatible cleavable polymer precursor, a biocompatible non-cleavable polymer precursor, and a photoinitiator.

2. The bioink formulation of claim 1, wherein the non-cleavable polymer precursor is gelatin methacryloyl (GelMA).

3. The bioink formulation of claim 2, wherein the GelMA has a concentration ranging from 2.5% to 7.5% by weight.

4. The bioink formulation of claim 1, wherein the cleavable polymer precursor is a hyaluronic acid derivative.

5. The bioink formulation of claim 4, wherein the molecular weight (Mw) of the hyaluronic acid derivative ranges from about 10 kDa to about 1,500 kDa.

6. The bioink formulation of claim 4, wherein the M_{w} of the hyaluronic acid derivative ranges from about 75 kDa to about 125 kDa.

7. The bioink formulation of claim 4, wherein the hyaluronic acid derivative is hyaluronic acid methacrylate (HAMA).

8. The bioink formulation of claim 7, wherein the HAMA has a concentration ranging from 0.5% to 5% by weight.

9. The bioink formulation of claim 1, wherein the non-cleavable polymer is GelMA and the cleavable polymer is HAMA.

10. The bioink formulation of claim 9, wherein the GelMA has a concentration ranging 2% to 6% by weight, and the HAMA has a concentration ranging from 1% to 2.5% by weight.

11. The bioink formulation of claim 1, wherein the bioink formulation further comprises cells.

12. A method of three-dimensional (3D) bioprinting, comprising:
providing a biocompatible cleavable polymer precursor, a biocompatible non-cleavable polymer precursor, and a photoinitiator in a 3D bioprinter vat;
repeatedly photoactivating the biocompatible polymer precursors in the 3D bioprinter vat on a build plate immersed in the vat to form a 3D bioprinted object comprising polymers having a series of predefined shapes across the vertical direction based on a set of sliced images; and
treating the 3D bioprinted object with an agent that cleaves chemical bonds within the cleavable polymer.

13. The method of claim 12, wherein the 3D bioprinted object is a tissue construct.

14. The method of claim 12, wherein the agent is an enzyme.

15. The method of claim 14, wherein the enzyme is a glycosidase or a protease.

16. The method of claim 12, wherein the non-cleavable polymer precursor is gelatin methacryloyl (GelMA).

17. The method of claim 12, wherein the cleavable polymer precursor is hyaluronic acid methacrylate (HAMA).

18. The method of claim 12, wherein the 3D bioprinted object further comprises cells.

19. The method of claim 13, wherein the tissue construct is a liver tissue construct and the mix of polymers further comprises hepatocytes.

20. The method of claim 13, wherein the tissue construct is a muscle tissue construct and the mix of polymers further comprises myoblasts.

21. The method of claim 13, wherein the tissue construct is a brain tissue construct and the mix of polymers further comprises neural progenitor cells.
